(19) United States
(12) Patent Application Publication
(10) Pub. No.: US 2024/0374203 A1
(43) Pub. Date: Nov. 14, 2024

(54) COGNITIVE FUNCTION EVALUATION METHOD, COGNITIVE FUNCTION INSPECTION METHOD, SYSTEM, AND COMPUTER PROGRAM

(71) Applicant: MAXELL, LTD., Kyoto (JP)

(72) Inventor: Tomohiko MIZUGUCHI, Kyoto (JP)

(21) Appl. No.: 18/696,171

(22) PCT Filed: Sep. 26, 2022
(86) PCT No.: PCT/JP2022/035750

§ 371 Date: Mar. 27, 2024

(30) Foreign Application Priority Data
Sep. 28, 2021 (JP) 2021-158099

(51) Int. Cl. A61B 5/00; A61B 5/11

(52) U.S. Cl. CPC A61B 5/4088; A61B 5/1124; A61B 5/7435

(57) ABSTRACT

A cognitive function evaluation method includes executing an inspection based on a TMT for urging a subject to connect passing points by a line, extracting, from the obtained inspection data, first inspection value data related to a residence time during which a line drawn by the subject sequentially tracking the passing points stagnates at a first passing point that is any one of the passing points from when the drawn line reaches the first passing point until when the drawn line starts moving to a second passing point, and second inspection value data related to a movement time required from when the drawn line starts moving from the first passing point until when the drawn line reaches the second passing point; and evaluating a cognitive function of the subject based on the first inspection value data and the second inspection value data which have been extracted.

### TECHNICAL FIELD

The present invention relates to a cognitive function evaluation method, a cognitive function inspection method, system, and computer program capable of evaluating a cognitive function based on a trail making test (TMT) inspection.

### BACKGROUND ART

In recent years, dementia has become a serious concern with an aging society. Generally, dementia is recognized as a disease in which symptoms progress with age and it is difficult to clearly grasp the onset time. In addition, it is also known that, although it is difficult to recover from dementia once dementia has actually developed and symptoms progress, it is possible to prevent the appearance of dementia by paying attention to usual lifestyle or the like, or it is possible to delay the progress of dementia by dealing therewith appropriately at the early stage of appearance of dementia. Therefore, nowadays, for early diagnosis and early measures of dementia, various screening inspections capable of early diagnosing the risk of dementia have been proposed and implemented.

As one example of such screening inspections, a trail making test (hereinafter, simply referred to as TMT through the entire specification) is known (see, for example, Patent Literature 1). This TMT is an examination for comprehensively measuring attention, working memory, spatial search, processing speed, persistence, impulsivity, and the like in a wide range by urging a subject to connect numbers and alphabets (or hiragana) randomly written on paper in order by a line.

Specifically, the TMT inspection includes a TMT-A inspection, a TMT-B inspection, and a TMT-J inspection. In the TMT-A inspection, a paper having numbers from 1 to 25 randomly arranged based on a predetermined rule is used, and the numbers should be connected in order by a line from 1 to 25 using a writing instrument and the inspection is ended at a time point when the line reaches 25. A time required for ending the inspection is measured. On the other hand, in the TMT-B inspection, a paper having 13 numbers from 1 to 13 and 12 alphabets from A to L or hiragana characters (a, i, u . . . ; i, ro, ha . . . or the like) corresponding thereto are randomly arranged based on a predetermined rule is used, the numbers and the alphabets (hiragana) should be alternately connected in order by a line using a writing instrument. Similarly, a time required for ending the inspection is measured. In the TMT-J inspection, several patterns having arrangement forms of numbers and alphabets, or the like different from those in the TMT-A inspection and the TMT-B inspection are prepared, and the similar inspection is performed based thereon.

Then, in such a TMT inspection, it is evaluated that the shorter the time required for ending the inspection (time required from start to end of the TMT; hereinafter, referred to as an implementation time) is, the faster the processing speed is and the longer the attention and concentration are retained. In addition, in the TMT, an actual inspection is always performed after a practice corresponding thereto before the inspection.

### CITATION LIST

### Patent Literature

Patent Literature 1: JP 2017-144252 A

### SUMMARY OF INVENTION

### Technical Problem

By the way, it has been found that such a TMT has a significant correlation with other known cognitive function tests such as the Mini-Mental State Examination (hereinafter, simply referred to as MMSE through the entire specification). Therefore, for example, in the correlation between the TMT and the MMSE, an attempt has been traditionally made to estimate the MMSE score (score that is obtained as an inspection result of the MMSE) based on the implementation time of the TMT. In this manner, by estimating the score of the MMSE, which generally takes longer time than the TMT, from the TMT inspection result, it is possible to reduce a time required for the cognitive examination.

However, it is hard to say that the estimation accuracy of the MMSE score based on the implementation time of the TMT is high. For example, it has been found, even from an examination of a correlation coefficient (for example, a value of R-square K-Fold) through a stepwise analysis (for example, a multiple regression analysis using k-Fold Cross Validation and cross validation with a R square value is applied), that a highly positive correlation cannot be obtained unless the number of times of TMT inspection is increased by repeating the TMT inspection many times. That is, an attempt to estimate the MMSE score based on the implementation time of the TMT requires an increasing number of times of TMT execution required for estimation, and thus it is difficult to obtain an estimation evaluation value of the cognitive function with high accuracy in a short time.

The present invention has been made in view of the above-described circumstances, and aims to provide a cognitive function evaluation method, a cognitive function inspection method, system, and computer program capable of executing a high-accuracy cognitive function evaluation with a high correlation between a TMT and an MMSE in a short time and also improving an MMSE score estimation accuracy.

### Solution to Problem

In order to solve the above-described problem, a cognitive function evaluation method according to the present invention includes:
a TMT execution step for executing an inspection based on a TMT for urging a subject to connect a plurality of passing points by a line such that the line passes through the passing points sequentially based on a predetermined rule;
a data extraction step for extracting, from inspection data obtained through the TMT execution step, first inspection value data related to a residence time during which a drawn line drawn by the subject sequentially tracking the passing points stagnates at a first passing point that is any one of the plurality of passing points from when the drawn line reaches the first passing point until when the drawn line starts moving to a second passing point to be passed through next, and second inspection value data related to a movement time required from when the drawn line starts moving from the first passing point until when the drawn line reaches the second passing point, for all of the passing points to be passed through; and
an evaluation step for evaluating a cognitive function of the subject based on the first inspection value data and the second inspection value data which have been extracted in the data extraction step.

The inventor of the present invention has reviewed the estimation of the MMSE score based on the implementation time of the TMT, which has been conventionally performed and in which the number of times of TMT to be performed is large and it is hard to say that the estimation accuracy is high, and has repeated trial and error to search for a parameter other than the implementation time which enhances the degree of a correlation between the TMT and the MMSE. Specifically, various parameters were selected as variables in a stepwise analysis (a multiple regression analysis using k-Fold Cross Validation, cross validation with a R square value is applied) in order to examine a correlation coefficient (a value of R-square K-Fold). As a result, four parameters having a high correlation between the TMT and the MMSE have been identified. That is, it has been found that, when a “residence time”, a “movement time”, a “position”, and a “direction” in the TMT were selected as parameters for evaluating the cognitive function, it was possible to perform a high-accuracy cognitive function evaluation with a high correlation between the TMT and the MMSE in a short time and improve the MMSE score estimation accuracy. Here, the “residence time” denotes, in an inspection based on a TMT for urging a subject to connect a plurality of passing points by a line such that the line passes through the passing points sequentially based on a predetermined rule, a time during which a drawn line drawn by the subject sequentially tracking the passing points stagnates at a first passing point that is any one of the plurality of passing points from when the drawn line reaches the first passing point until when the drawn line starts moving to a second passing point to be passed through next. Besides, the “movement time” denotes a time required from when the drawn line starts moving from the first passing point until when the drawn line reaches the second passing point. Furthermore, the “position” denotes a position of each passing point, and the “direction” denotes a movement direction directed from the first passing point to the second passing point.

In fact, in the above-described stepwise analysis, when a correlation coefficient that is a value of the R-square K-Fold was examined while adding thereto the “residence time”, the “movement time”, the “position”, and the “direction” as cognitive function evaluation parameters in a stepwise manner, a verification result that the degree of correlation between the TMT and the MMSE increases as the parameters to be added are gradually increased, has been obtained. Specifically, in the stepwise analysis, in a case where only the “residence time” and the “movement time” were added as the cognitive function evaluation parameters and the “position” and the “direction” were not added, the correlation coefficient (a value of R-square K-Fold) increased as the number of times of TMT execution increased, and it was possible to obtain a high degree of the correlation with a smaller number of times of TMT execution than the conventional number of times of TMT execution required for estimating the MMSE score based on the implementation time. In addition thereto, when the “position” and the “direction” were further added as the cognitive function evaluation parameters, and the stepwise analysis was performed based on the four cognitive function evaluation parameters: the “residence time”, the “movement time”, the “position”, and the “direction”, a higher degree of the correlation (a high correlation coefficient value exceeding 0.5) was obtained with a smaller number of times of TMT execution than a case where the “position” and the “direction” were not added.

Accordingly, in the cognitive function evaluation method having the above-described configuration according to the present invention, the first inspection value data related to the “residence time” and the second inspection value data related to the “movement time” are extracted, from the inspection data obtained through the TMT execution step for executing an inspection based on the TMT, for all the passing points to be passed through, and the subject's cognitive function is evaluated based on the first inspection value data and the second inspection value data which have been extracted. Accordingly, the correlation between the TMT and the MMSE increases, and the number of times of TMT execution required for estimating the MMSE score decreases, whereby a highly accurate cognitive function evaluation can be performed in a short time. Besides, in addition to the first and second inspection value data, the position data related to the “position” of the passing point and the movement direction data related to the “movement direction” between the passing points are extracted for all the passing points to be passed through, and the subject's cognitive function is evaluated based on the first inspection value data, the second inspection value data, the position data, and the movement direction data which have been extracted, whereby it becomes possible to execute, in a shorter time, a highly accurate cognitive function evaluation in which the correlation between the TMT and the MMSE further increases. Such a cognitive function evaluation is effective as a screening in an initial stage for judging dementia, and has a wide range of application: as a matter of course, it helps the detection of dementia, and can also contribute to the determination of a judgment, for example, in driving a car, and can be applied to a brain training type game, for example.

In the above-described configuration of the present invention, the cognitive function evaluation method further includes a characteristic diagram generation step for generating a characteristic diagram in which the first inspection value data or the second inspection value data, and the position data or the movement direction data are displayed in association with each other, and the cognitive function may be evaluated based on the characteristic diagram generated in the characteristic image generation step. According to this, since the generated characteristic diagram displays the inspection value and the position data or the movement direction data in association with each other, it is possible to visually grasp at a glance how the residence time and the movement time change for the position of each passing point or for the movement direction in each area between the passing points, and thus, it is possible to allow a doctor to perform a fine cognitive function evaluation, for example, and to provide a useful display form that helps the cognitive function evaluation.

The present invention also provides a cognitive function evaluation system to which the above-mentioned cognitive function evaluation method is applied. That is, the present invention is a cognitive function evaluation system that enables a TMT inspection on a display, evaluates a result of the inspection, and allows the result to be displayed on the display, including:
an inspection image generation circuit that electronically generates a TMT inspection image displayed on the display and obtained by setting passing points at a plurality of positions on a coordinate plane;
an inspection data acquisition circuit that acquires chronological data of a drawing trajectory drawn by a subject moving a contactor in a state of being in contact with a display surface of the TMT inspection image on the display so as to track the passing points in a predetermined order;
a data processing circuit that processes the data acquired by the inspection data acquisition circuit and makes a result of the processing displayable on the display as the result of the inspection; and
a control circuit that controls an operation of each of the circuits, wherein
the inspection data acquisition circuit includes a coordinate data acquisition circuit that acquires coordinate data corresponding to a position of the contactor on the coordinate plane based on a detection signal from a sensor that detects the contact of the contactor with the display surface of the TMT inspection image, and a time data acquisition circuit that acquires time data associated with an acquisition time of each piece of the coordinate data by a timer, and
the data processing circuit includes a computation circuit that computes, based on the coordinate data and the time data in the contactor, a first inspection value related to a residence time during which the contactor stagnates at a first passing point that is any one of the plurality of passing points from when the contactor reaches the first passing point until when the contactor starts moving to a second passing point to be passed through next, and a second inspection value related to a movement time required from when the contactor starts moving from the first passing point until when the contactor reaches the second passing point, for all of the passing points to be passed through, an evaluation circuit that evaluates a cognitive function of the subject based on the first inspection value and the second inspection value, and an inspection result outputting circuit that outputs the result of the inspection including a result of the evaluation executed by the evaluation circuit.

Also in such a cognitive function evaluation system, similarly to the cognitive function evaluation method described above, the subject's cognitive function is evaluated based on the first inspection value related to the “residence time” and the second inspection value related to the “movement time”, and thus, a correlation between the TMT and the MMSE increases and the number of times of TMT execution required for estimating the MMSE score is reduced, whereby a highly accurate cognitive function evaluation can be performed in a short time. Also in this case, in addition to the first and second inspection values, the position data related to the “position” of the passing point and the movement direction data related to the “movement direction” between the passing points are acquired, and the subject's cognitive function is evaluated based on the first inspection value data, the second inspection value data, the position data, and the movement direction data, whereby it becomes possible to execute, in a shorter time, a highly accurate cognitive function evaluation in which the correlation between the TMT and the MMSE further increases.

Also in the cognitive function evaluation system having the above-described configuration, it is preferable that the characteristic image generation circuit generates a characteristic image (corresponding to the characteristic diagram in the above-described cognitive function evaluation method) that displays the first inspection value or the second inspection value, and the position data or the movement direction data are displayed in association with each other, and the evaluation circuit evaluates the cognitive function based on the characteristic image. According to this, since the generated characteristic image displays the inspection value and the position data or the movement direction data in association with each other, it is possible to visually grasp at a glance how the residence time and the movement time change for the position of each passing point or for the movement direction in each area between the passing points, and thus, it is possible to allow a doctor to perform a fine cognitive function evaluation, for example, and to provide a useful display form that helps the cognitive function evaluation.

In addition, in such a cognitive function evaluation system, the following operation is also possible. That is, for example, when an inspection form of the TMT inspection is selected and inputted from a mode selection menu displayed on the display, the inspection image generation circuit generates a TMT inspection image (for example, an image for the TMT-A inspection, an image for the TMT-B inspection, an image for the TMT-J inspection, or the like) according to the selected inspection form under the control of the control circuit, and the TMT inspection image is displayed on the display. Then, when the subject moves the contactor while bringing the contactor into contact with the display surface of the displayed TMT inspection image so as to track the passing points in a predetermined order, the chronological data of the drawing trajectory drawn thereby is acquired by the inspection data acquisition circuit. The data acquired by the inspection data acquisition circuit is processed by the data processing circuit, and a result of the processing including the evaluation of the subject's cognitive function is, once a display form of the inspection result is selected and inputted from the mode selection menu, dynamically and/or statically displayed, as the inspection result, on the display by the data processing circuit in the selected display form under the control of the control circuit, for example. As described above, according to the cognitive function evaluation system of the present invention, since a series of processes from the execution of the inspection until the acquisition of the inspection result (the display of the inspection result) can be automated, it is not necessary for an inspector to measure the time required for the inspection with a stopwatch or the like, and it is not necessary to manually count and analyze the obtained inspection data including the measurement value. Therefore, a series of processes from the execution of the inspection until the acquisition of the inspection result (the display of the inspection result) can be quickly and easily performed.

In addition thereto, in the cognitive function evaluation system of the present invention, the inspection data acquisition circuit includes a coordinate data acquisition circuit that acquires coordinate data corresponding to a position of the contactor on the coordinate plane based on a detection signal from a sensor that detects the contact of the contactor with the display surface of the TMT inspection image, and a time data acquisition circuit that acquires time data associated with an acquisition time of each piece of the coordinate data by a timer, and the data processing circuit includes a computation circuit that computes, based on the coordinate data and the time data in the contactor, the first and second inspection values related to the “residence time” and the “movement time”, an evaluation circuit that evaluates a cognitive function of the subject based on the first inspection value and the second inspection value, and an inspection result outputting circuit that outputs the result of the inspection including a result of the evaluation by the evaluation circuit. That is, the cognitive function evaluation system of the present invention can acquire a temporal change in the position of the contactor on the coordinate plane as time-series coordinate data based on an electrical detection signal of a contact detection sensor, can acquire an elapsed time associated with the stagnation and the movement of the contactor as time data by the timer based on the detection signal from the contact detection sensor, and can compute the first and second inspection values, that is, the residence time and the movement time of the contactor, based on the above-described data, thereby automatically evaluating the subject's cognitive function. Therefore, it becomes possible to reliably capture various kinds of hidden information in the inspection process which cannot be obtained only from the measurement value and the drawing trajectory associated with the manual measurement by using a stopwatch or the like and the drawing by the subject, and to make the information useful for the evaluation of the subject's cognitive function by a doctor or the like. In addition, according to such an automatic inspection form accompanied with the electrical processing, it is possible to eliminate a human measurement error and unify an inspection condition, and thus, it is possible to prevent a situation in which inspection results fluctuate every time an inspection is performed, whereby the reliability of the inspection result can be improved.

Furthermore, according to such an automated cognitive function evaluation system, the subject can perform an inspection alone without an inspector and confirm the result instantly.

Note that, in the above-described configuration, the “contactor” may be anything as long as it can be moved in a state of being in contact with the display surface of the TMT inspection image so as to draw a drawing trajectory and is a broad concept including not only an electronic input device such as a stylus pen operated by the subject, but also the subject's finger. In the above configuration, the “sensor” may be based on any detection principle as long as it can detect a contact position of the contactor with respect to the display surface of the TMT inspection image, and may be provided on the contactor side or may be provided on the display surface side. Furthermore, the above-described various circuits whose operations are controlled by the control circuit may be provided as physically individual circuits. However, a functional unit (or an apparatus) that integrates at least a part of or the entire of these circuits may be configured (for example, may be electronically put in a single package). In short, the circuits may exist in any form as long as the functions of these respective circuits are maintained.

Note that it is preferable that the cognitive function evaluation system having the above-described configuration further includes a memory that stores therein the inspection data including the coordinate data and the time data and the characteristic image generated by the characteristic image generation circuit. According to this, it is possible to accumulate data in the memory and read out required data in a timely manner as necessary. In addition, for example, it becomes possible to evaluate the progress of symptoms by comparing pieces of the accumulated history data, or execute a final authorization of the evaluation based on the data accumulated in the memory. Here, the “inspection data” means all unprocessed data that can be acquired by the inspection data acquisition circuit.

In addition, in the above-described configuration, it is preferable that the characteristic image generation circuit includes an identification image generation circuit that classifies the position data or the movement direction data into a plurality of groups based on the position or the direction on the coordinate plane, and generates a characteristic image in such a display form that inspection values corresponding to the respective groups are visually identifiable from one another.

Here, the “display form that . . . are visually identifiable” is a display form in which data (inspection values) corresponding to the respective groups can be visually distinguished from each other at a glance depending on, for example, differences in color, line type, pattern, or the like. In addition, the grouping can be selected by a system user including an inspector and a subject, for example, through the mode selection menu, and the control circuit controls the characteristic image generation circuit based on an input signal from the mode selection menu associated with the selection to generate an identifiably displayed image.

According to such an identifiably displaying function, for example, a coordinate plane is divided vertically and horizontally into four regions, that is, the coordinate plane is divided into a first quadrant, a second quadrant, a third quadrant, and a fourth quadrant. Then, data (inspection values) corresponding to the respective quadrants are displayed in different graphs (for example, a bar graph or a circular graph), or are color-coded in four colors in the same graph. Alternatively, by setting a plurality of (for example, eight) boundary straight lines that extends radially from a coordinate origin and are separated from one another at equal angular intervals around the coordinate origin, the data is displayed in an identifiable manner by specifying a plurality of (for example, eight) regions defined by these boundary straight lines in the coordinate plane, and by color-coding the data (inspection values) corresponding to the respective regions in the number of colors (for example, eight colors) corresponding to the number of regions. Alternatively, 360-degree direction is divided into some directional groups, and data (inspection values) corresponding to the respective groups is displayed in an identifiable manner by being displayed in different graphs (for example, a bar graph or a circular graph) or are color-coded in four colors in the same graph. Therefore, it is possible to grasp a tendency unique to each inspection value according to the display form thereof at a glance. For example, it is possible to clearly grasp, visually at a glance, a tendency of an inspection result depending on whether the dominant hand of a subject is a left hand or a right hand, a tendency of an inspection result caused by a damaged site in the brain, and the like, or a tendency of an inspection result associated with, for example, the visual loss of the eye on one side or a partial or entire decline in the physical function. Therefore, it is possible to facilitate the evaluation of the subject's cognitive function by a doctor or the like.

In the above-described configuration, the computation circuit may compute sums of the inspection values for the respective groups, and the identification image generation circuit may generate a characteristic image that displays the sums for the respective groups. Alternatively, the computation circuit may compute sums of the inspection values for the respective groups and compute rates of the sums of the respective groups with respect to a sum of all groups, and the identification image generation circuit generates a characteristic image that displays the rates for the respective groups. According to this, it is possible to grasp, at a glance, a tendency unique to each group and also grasp the degree of tendency of each group with respect to all groups, which can contribute to a simple evaluation of the cognitive function.

Besides, the cognitive function evaluation system having the above-described configuration may further includes a passage detection circuit that detects passage of the contactor at the passing point based on a detection signal from the sensor, wherein: the passage detection circuit sets, for each passing point, a first coordinate region for determining that the contactor has passed through the passing point and a second coordinate region for determining that the contactor stagnates at the passing point, detects entry into the first coordinate region of the contactor as the passage of the contactor at the passing point, detects movement of the contactor in the second coordinate region as the stagnation of the contactor at the passing point, and detects movement of the contactor from the inside of the second coordinate region to the outside of the second coordinate region as the movement of the contactor from the passing point; and the computation circuit computes the first inspection value and the second inspection value based on signals indicating the stagnation and the movement from the passage detection circuit.

With such a configuration, the passage detection circuit can recognize a state (a fluctuating state of the contactor) in which the subject is searching for the following second passing point while performing an operation of moving the contactor to some extent in the second coordinate region after moving the contactor to enter the first coordinate region at the first passing point, as a state in which the contactor stagnates at the first passing point, and can recognize that, when the contactor has moved from the inside of the second coordinate region to the outside of the second coordinate region, the contactor has moved away from the first passing point toward the second passing point. That is, by clearly defining a boundary between a stagnation state and a movement state in this manner to eliminate an ambiguous state in which it is unclear whether or not the contactor has moved from the first passing point, an automatic determination of the “stagnation” and the “movement” can be reliably performed, and the “residence time” and the “movement time” can be accurately calculated.

In addition, in the above-described configuration, it is preferable that the passage detection circuit includes a setting circuit for variably setting the ranges of the first coordinate region and the second coordinate region. According to this, the setting circuit can set allowable ranges related to the determination of whether or not the contactor has passed through the passing point, whether or not the contactor is stagnating at the passing point, and whether or not the contactor has moved from the passing point. In this case, it is possible to give free rein to the determination of passage, stagnation, and movement, whereas it is also possible to impose a certain constraint on the free rein by defining the variable setting range with coordinates, whereby an inspection having a degree of freedom depending on situations is enabled while minimizing the fluctuation of inspection results caused by the free rein.

The present invention also provides a cognitive function inspection method including:
a TMT execution step for executing an inspection based on a TMT for urging a subject to connect a plurality of passing points by a line such that the line passes through the passing points sequentially based on a predetermined rule;
a data extraction step for extracting, from inspection data obtained through the TMT execution step, first inspection value data related to a residence time during which a drawn line drawn by the subject sequentially tracking the passing points stagnates at a first passing point that is any one of the plurality of passing points from when the drawn line reaches the first passing point until when the drawn line starts moving to a second passing point to be passed through next, and second inspection value data related to a movement time required from when the drawn line starts moving from the first passing point until when the drawn line reaches the second passing point, for all of the passing points to be passed through; and
a data processing and displaying step for processing the first inspection value data and the second inspection value data which have been extracted in the data extraction step and displaying a result of the processing as an inspection result.

In such a cognitive function inspection method, inspection value data are processed and a result of the processing is displayed as an inspection result. Thus, this method can contribute to a doctor's appropriate determination regarding the cognitive function.

In addition to the above-described cognitive function evaluation method and system, the present invention also provides a method and a computer program suitable for an automatic evaluation of the cognitive function.

### Advantageous Effects of Invention

According to the present invention, at least the first inspection value related to the “residence time” and the second inspection value related to the “movement time” are obtained, for all of the passing points to be passed through, from the inspection data obtained through the execution of the TMT, and the subject's cognitive function is evaluated based on these inspection values. Therefore, it is possible to perform a high-accuracy cognitive function evaluation with a high correlation between the TMT and the MMSE in a short time and also improve the MMSE score estimation accuracy.

### BRIEF DESCRIPTION OF DRAWINGS

FIG. 1 is a flowchart illustrating a conceptual flow of a cognitive function evaluation method according to the present invention.

FIG. 2 is a block diagram illustrating a configuration of a cognitive function evaluation system according to one embodiment of the present invention.

FIG. 3 is a flowchart schematically illustrating a flow of a process of executing a TMT inspection using the cognitive function evaluation system in FIG. 2 and displaying the inspection result.

FIG. 4(a) illustrates one example of a TMT inspection image for a TMT-A inspection displayed on a display, and FIG. 4(b) illustrates one example of a TMT inspection image for a TMT-B inspection displayed on the display.

FIG. 5 illustrates one example of a display setting screen displayed on the display.

FIG. 6(a) illustrates one example of a TMT inspection image when a direction of a vertical version is selected on the display setting screen in FIG. 5, and FIG. 6(b) illustrates one example of a TMT inspection image when a direction of a horizontal version is selected on the display setting screen in FIG. 5.

FIG. 7(a) illustrates one example of a TMT inspection image when a pattern A is selected on the display setting screen in FIG. 5, FIG. 7(b) illustrates one example of a TMT inspection image when a pattern B is selected on the display setting screen in FIG. 5, FIG. 7(c) illustrates one example of a TMT inspection image when a pattern C is selected on the display setting screen in FIG. 5, and FIG. 7(d) illustrates one example of a TMT inspection image when a pattern D is selected on the display setting screen in FIG. 5.

In a display form in which a display line of a drawing trajectory drawn by a subject on a display surface of a TMT inspection image is gradually thickened from the first stage to the fifth stage, FIG. 8(a) illustrates a drawing trajectory on a TMT inspection image when the thickness of the display line is set to the first stage on the display setting screen in FIG. 5, FIG. 8(b) illustrates a drawing trajectory on the TMT inspection image when the thickness of the display line is set to the second stage on the display setting screen in FIG. 5, FIG. 8(c) illustrates a drawing trajectory on the TMT inspection image when the thickness of the display line is set to the third stage on the display setting screen in FIG. 5, FIG. 8(d) illustrates a drawing trajectory on the TMT inspection image when the thickness of the display line is set to the fourth stage on the display setting screen in FIG. 5, and FIG. 8(e) illustrates a drawing trajectory on the TMT inspection image when the thickness of the display line is set to the fifth stage on the display setting screen in FIG. 5.

FIG. 9(a) illustrates a drawing trajectory on a TMT inspection image when the setting is performed on the display setting screen in FIG. 5 such that the color of a passing point changes when a contactor comes into contact with the passing point (has passed through the passing point), and FIG. 9(b) illustrates a drawing trajectory on a TMT inspection image when the setting is performed on the display setting screen in FIG. 5 such that the color of a passing point does not change when the contactor comes into contact with the passing point (has passed through the passing point).

FIG. 10(a) illustrates a TMT inspection image when the setting is performed on the display setting screen in FIG. 5 such that a drawing trajectory of a subject is not visually displayed, FIG. 10(b) illustrates a TMT inspection image when the setting is performed on the display setting screen in FIG. 5 such that a drawing trajectory of a subject is visually displayed only in an area between the latest two passing points (between a passing point which has been lastly passed at the present time during the drawing and a passing point which has been passed immediately therebefore), and FIG. 10(c) illustrates a TMT inspection image when the setting is performed on the display setting screen in FIG. 5 such that a drawing trajectory of a subject is visually displayed among all passing points.

FIG. 11(a) is a diagram illustrating a reference region, a first coordinate region, and a second coordinate region set for each passing point, and FIG. 11(b) is a diagram illustrating one example of a stagnation trajectory tracked by the contactor at a first passing point and a movement trajectory tracked by the contactor in an area between the first passing point and a second passing point.

FIG. 12(a) is a diagram illustrating a coordinate plane of a TMT inspection image divided vertically and horizontally into four regions (quadrants), and FIG. 12(b) is a diagram illustrating four directions on the coordinate plane in FIG. 12(a).

FIG. 13 is a diagram illustrating one example of a processed image in which a first inspection value, a second inspection value, position data, and movement direction data are displayed in a table form.

FIG. 14(a) is a diagram illustrating one example of a characteristic image in which a residence time that is the first inspection value and a position of a passing point are displayed in association with each other for all passing points, and FIG. 14(b) is a diagram illustrating one example of a characteristic image in which a residence time that is the first inspection value and a movement direction between passing points are displayed in association with each other for all passing points.

FIG. 15(a) is a diagram illustrating one example of a characteristic image in which a movement time that is the second inspection value and a position of a passing point are displayed in association with each other for all passing points, and FIG. 15(b) is a diagram illustrating one example of a characteristic image in which a movement time that is the second inspection value and a movement direction between passing points are displayed in association with each other for all passing points.

FIG. 16(a) is a diagram illustrating one example of a characteristic image in which a residence time that is the first inspection value and a position of a passing point are displayed in each quadrant in association with each other for all passing points, and FIG. 16(b) is a diagram illustrating one example of a characteristic image in which a residence time that is the first inspection value and a movement direction between passing points are displayed in each quadrant in association with each other for all passing points.

FIG. 17(a) is a diagram illustrating one example of a characteristic image in which a movement time that is the second inspection value and a position of a passing point are displayed in each quadrant in association with each other for all passing points, and FIG. 17(b) is a diagram illustrating one example of a characteristic image in which a movement time that is the second inspection value and a movement direction between passing points are displayed in each quadrant in association with each other for all passing points.

FIG. 18 is a diagram illustrating one example of a characteristic image in which the first inspection value pertaining to a residence time and a position of a passing point are displayed in association with each other: FIG. 18(a) is a diagram in which the sums of the residence times for the respective quadrants are displayed in an identifiable manner using a bar graph; FIG. 18(b) is a diagram in which the sums of the residence times for the respective quadrants are displayed in different colors or different patterns in a circular graph; FIG. 18(c) is a diagram in which the sums of the residence times for the respective quadrants are displayed in an identifiable manner using a bar graph; and FIG. 18(d) is a diagram in which the sums of the residence times for the respective quadrants are displayed in different colors or different patterns in a band graph.

FIG. 19 is a diagram illustrating one example of a characteristic image in which the first inspection value pertaining to a residence time and a position of a passing point are displayed in association with each other: FIG. 19(a) is a diagram in which the rates (%) of the residence times for the respective quadrants are displayed in an identifiable manner using a bar graph; FIG. 19(b) is a diagram in which the rates of the residence times for the respective quadrants are displayed in different colors or different patterns in a circular graph; FIG. 19(c) is a diagram in which the rates of the residence times for the respective quadrants are displayed in an identifiable manner using a bar graph; and FIG. 19(d) is a diagram in which the rates of the residence times for the respective quadrants are displayed in different colors or different patterns in a band graph.

FIG. 20 is a diagram illustrating one example of a characteristic image in which the first inspection value pertaining to a residence time and a movement direction between passing points are displayed in association with each other: FIG. 20(a) is a diagram in which the sums of the residence times for the respective movement directions are displayed in an identifiable manner using a bar graph; FIG. 20(b) is a diagram in which the sums of the residence times for the respective movement directions are displayed in different colors or different patterns in a circular graph; FIG. 20(c) is a diagram in which the sums of the residence times for the respective movement directions are displayed in an identifiable manner using a bar graph; and FIG. 20(d) is a diagram in which the sums of the residence times for the respective movement directions are displayed in different colors or different patterns in a band graph.

FIG. 21 is a diagram illustrating one example of a characteristic image in which the first inspection value pertaining to a residence time and a movement direction between passing points are displayed in association with each other: FIG. 21(a) is a diagram in which the rates (%) of the residence times for the respective movement directions are displayed in an identifiable manner using a bar graph; FIG. 21(b) is a diagram in which the rates of the residence times for the respective movement directions are displayed in different colors or different patterns in a circular graph; FIG. 21(c) is a diagram in which the rates of the residence times for the respective movement directions are displayed in an identifiable manner using a bar graph; and FIG. 21(d) is a diagram in which the rates of the residence times for the respective movement directions are displayed in different colors or different patterns in a band graph.

FIG. 22 is a conceptual diagram illustrating a state in which a terminal as a TMT inspection result display system according to one embodiment of the present invention is connected to a server via communication means.

### DESCRIPTION OF EMBODIMENTS

Hereinafter, the embodiment of the present invention will be described with reference to the drawings.

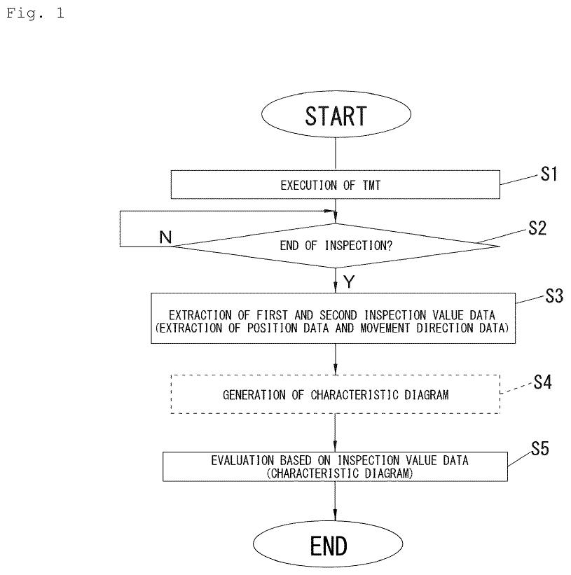

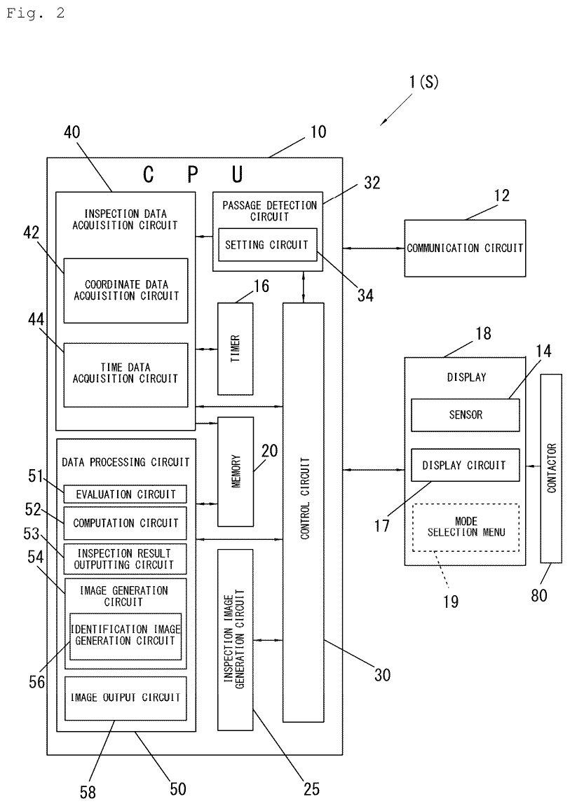

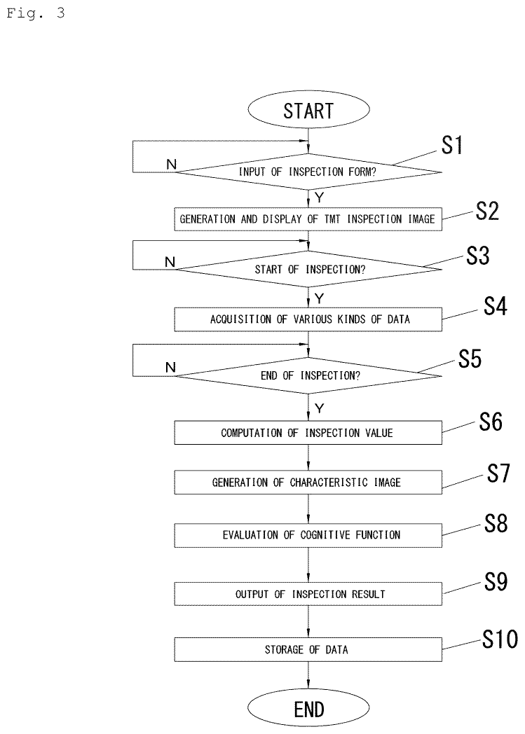

FIG. 1 illustrates a conceptual flow of a cognitive function evaluation method according to the present invention. As illustrated in the drawing, in the cognitive function evaluation method of the present invention, firstly, an inspection is executed based on a known TMT for urging a subject to connect a plurality of passing points by a line such that the line passes through the passing points sequentially based on a predetermined rule (TMT execution step S1). In the inspection based on the TMT, at least the time when a drawn line drawn by the subject sequentially tracking the passing points reaches each passing point, the time when the drawn line starts moving from each passing point, and the like are measured as inspection data, and the movement direction between the passing points, the positions of the passing points, and the like are also recorded. Then, once the inspection based on the TMT is completed (in a case of YES in step S2), next, from the obtained inspection data, first inspection value data related to a residence time during which a drawn line drawn by the subject sequentially tracking the passing points stagnates at a first passing point that is any one of the plurality of passing points from when the drawn line reaches the first passing point until when the drawn line starts moving to a second passing point to be passed through next, and second inspection value data related to a movement time required from when the drawn line starts moving from the first passing point until when the drawn line reaches the second passing point, are extracted (acquired), for all of the passing points to be passed through (data extraction step S3). In this case, from the inspection data, the position data related to the positions of the first passing point and the second passing point as well as the movement direction data related to the movement direction directed from the first passing point to the second passing point may be further extracted for all the passing points to be passed through.

Once the inspection value data is extracted in this manner, the subject's cognitive function is evaluated based on the first inspection value data and the second inspection value data which have been extracted (also based on the position data and/or the movement direction data) (evaluation step S5). Specifically, in such a cognitive function evaluation, for example, it is conceivable to set a cut-off value (threshold) corresponding to each measured value (computed value) related to the residence time at each passing point and the movement time between the respective passing points, and evaluate the subject's cognitive function by comparing the cut-off value with the measured value. In that case, for example, in order to classify the subjects into three groups: healthy people, people with MCI (Mild Cognitive Impairment), and dementia patients, a cut-off value may be set for each of these groups. Alternatively, in a case of evaluating the subject's cognitive function based on the first inspection value data, the second inspection value data, the position data, and the movement direction data, for example, as illustrated in FIG. 13 to be described later that is associated with a TMT inspection image obtained by setting passing points at a plurality of positions on a coordinate plane as illustrated in FIG. 12, measurement data (computation data) in which a residence time at each passing point and a movement time between the respective passing points are listed depending on the position (quadrant position) of the passing point and the movement direction between the passing points is acquired, and the measurement data is compared with a cut-off value set for the residence time and the movement time corresponding to each passing point, whereby subjects may be classified, for the evaluation, into, for example, three: healthy people, people with MCI (Mild Cognitive Impairment), and dementia patients. In this case, a total value of the measured values may be computed for each quadrant, and the computation result may be compared with a cut-off value corresponding to the total value. As described above, when the subject's cognitive function is evaluated based on the first inspection value data and the second inspection value data (also based on the position data and/or the movement direction data), as is apparent from the above-described results of verification by the inventors, a correlation between the TMT and the MMSE increases and the number of times of TMT execution required for estimating the MMSE score is reduced, whereby a highly accurate cognitive function evaluation can be performed in a short time.

In such evaluation, a characteristic diagram in which the first inspection value data or the second inspection value data, and the position data or the movement direction data are displayed in association with each other may be used. In such a case, a characteristic diagram is generated based on data extracted in the data extraction step S3 (characteristic diagram generation step S4), and in the evaluation step S5, the cognitive function is evaluated based on the characteristic diagram generated in the characteristic diagram generation step S4. Examples of the characteristic diagram include characteristic diagrams (characteristic images in an automation system to be described later) illustrated in FIGS. 14-21 to be described later. Specifically, in the cognitive function evaluation using such a characteristic diagram, the cognitive function may be evaluated by superimposing a characteristic diagram of the subject with a characteristic diagram of the healthy people (for example, an average characteristic diagram).

Furthermore, in the evaluation step S5, the MMSE score may be estimated as a cognitive function evaluation value of the subject based on the data extracted in the data extraction step S3. Specifically, such an estimation of the MMSE score may be calculated through calculation using a weighting function forming an N-th degree polynomial. That is, for example, in a case where a list of measurement data (computation data) as illustrated in FIG. 13 to be described later is acquired, the total inspection time, the residence time at each passing point (search time; first inspection value data), the movement time between the respective passing points (drawing line time; second inspection value data), the position of the passing point (quadrant position; position data) and the movement direction between the passing points (drawing line direction; movement direction data) are used as indices (parameters), and each of these indices is weighted and added, thereby calculating the MMSE score as estimated value in accordance with the following formula.

$$MMSE score = α1 \times total inspection time ( 1 - 25 ) + β1 \times SearchTime ( 2 ) + β2 \times Search Time ( 3 ) + ... + β24 \times \\ SearchTime ( 25 ) + γ1 \times DrawingTime ( 2 ) + γ2 \times DrawingTime ( 3 ) + ... + γ24 \times DrawingTime ( 25 ) + Δ1 \times quadrant ( 2 ) + Δ2 \times quadrant ( 3 ) + ... + Δ 24 \times quadrant ( 25 ) + E 1 \times drawing line direction ( 2 ) + E 2 \times drawing line direction ( 3 ) + ... + E 24 \times drawing line direction ( 25 )$$

Here, in the formula, numbers in parentheses are the number of the passing point (target circle number), and a, β, γ, Δ, and E are coefficients (multipliers).

Note that all the parameters may be added in this manner, but calculation may be performed with reduced number of parameters through dimension reduction.

The cognitive function evaluation method described above may be a method for supporting the cognitive function evaluation carried out by not only a party in charge of evaluation including a doctor and the like, but also a subject him/herself in various industrial fields, which are not limited to a medical field, or may be a method of detecting the cognitive state, and may be carried out through a manual operation or the like based on a standard TMT performed on paper. However, such an execution is very complicated and extremely difficult because an inspector such as a doctor or a person in charge of inspection needs to measure the time required for acquiring inspection data with a stopwatch or the like or needs to record inspection images or the like. In particular, it is troublesome to acquire the first to fourth inspection value data described above through a manual operation, and it also takes considerable efforts to aggregate and analyze the acquired data.

In order to eliminate such a drawback, an automated cognitive function evaluation system, computer program, and method capable of executing an inspection based on a TMT on a display as well as evaluating the inspection result and displaying the inspection result on the display will be described hereinafter.

The cognitive function evaluation system which enables a TMT inspection on the display and allows the inspection result to be displayed on the display is configured as a terminal as an example in the present embodiment, and as illustrated in FIG. 22, for example, such a terminal 1 (cognitive function evaluation system S) may be connected to a server 102 via communication means (network) 100 in a conceivable usage form (which will be described later), but any usage form may be adopted. Note that the terminal 1 is configured as a tablet-type thin computer in the present embodiment, but may be a personal computer, a smartphone, or the like.

In addition, in the present embodiment, the terminal 1 itself includes a display so that the TMT inspection and the display of inspection result (evaluation result) can be performed alone. However, the terminal 1 may be configured as a system that can perform the TMT test and the display of inspection result in cooperation with a separate display. Alternatively, the cognitive function evaluation system S may exist as a computer program that allows the TMT inspection and the display of inspection result to be performed by a computer or a computer program product in which the computer program is stored.

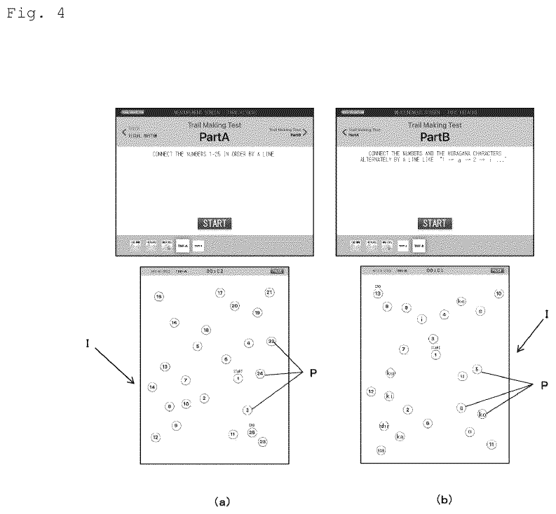

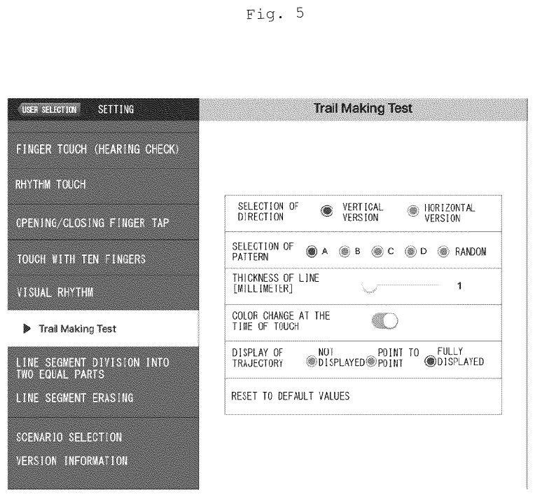

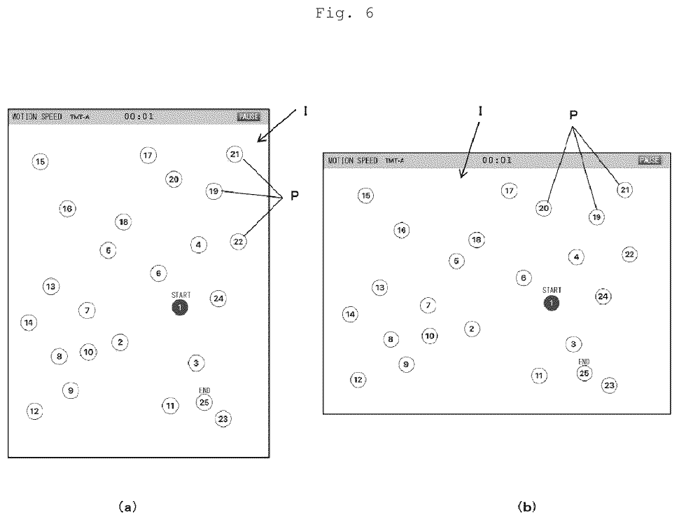

FIG. 2 is a block diagram illustrating a conceptual configuration of the terminal 1. As illustrated, the terminal 1 serving as the cognitive function evaluation system S includes, for example, a display 18 as a liquid crystal display and a CPU 10. The CPU 10 includes: an inspection image generation circuit 25 that electronically generates a TMT inspection image I (to be described later with reference to FIG. 4 and after) displayed on the display 18 and obtained by setting passing points P (to be described later with reference to FIG. 4 and after) at a plurality of positions on the coordinate plane; an inspection data acquisition circuit 40 that acquires chronological data of a drawing trajectory drawn by a subject moving a contactor 80 in a state of being in contact with a display surface of the TMT inspection image I so as to track the passing points P in a predetermined order; a data processing circuit 50 that processes the data acquired by the inspection data acquisition circuit 40 and makes a result of the processing displayable on the display 18 as an inspection result; and a control circuit 30 that controls the operation of each circuit 25, 40, and 50 based on an input signal from a mode selection menu 19 that is displayed on the display 18 and that allows an inspection form of the TMT inspection and a display form of the inspection result to be selected. Note that, in the present embodiment, the control circuit 30 controls a display circuit 17 provided on the display 18, thereby displaying various images on the display 18.

Furthermore, in the present embodiment, the contactor 80 is configured as an electronic input device such as a stylus pen operated by the subject, but may be anything as long as it can be moved in a state of being in contact with the display surface of the TMT inspection image I so as to draw a drawing trajectory and may be, for example, the subject's finger.

The inspection data acquisition circuit 40 includes a coordinate data acquisition circuit 42 that acquires coordinate data corresponding to the position of the contactor 80 on the coordinate plane based on a detection signal from a sensor 14 that detects the contact of the contactor 80 with the display surface of the TMT inspection image I, and a time data acquisition circuit 44 that acquires time data associated with an acquisition time of each piece of the coordinate data by a timer 16. In addition, the data processing circuit 50 includes: a computation circuit 52 that computes, based on the coordinate data and the time data in the contactor 80, a first inspection value V1 related to a residence time during which the contactor 80 stagnates at a first passing point P1 that is any one of the plurality of passing points P from when it reaches the first passing point P1 until when it starts moving to a second passing point P2 to be passed through next, and a second inspection value V2 related to a movement time required from when the contactor 80 starts moving from the first passing point P1 until when the contactor 80 reaches the second passing point P2, for all of the passing points P to be passed through; an evaluation circuit 51 that evaluates the cognitive function of the subject based on the first inspection value V1 and the second inspection value V2; and an inspection result outputting circuit 53 that outputs the inspection result including the result of evaluation executed by the evaluation circuit 51.

In addition, in the present embodiment, the inspection data acquisition circuit 40 may acquire the position data related to the positions of the first passing point P1 and the second passing point P2 as well as the movement direction data related to the movement direction directed from the first passing point P1 to the second passing point P2, for all of the passing points to be passed through. In response to this, the evaluation circuit 51 may evaluate the subject's cognitive function based on the first inspection value V1, the second inspection value V2, the position data, and the movement direction data.

Additionally, in the present embodiment, the data processing circuit 50 further includes: a characteristic image generation circuit 54 that generates a characteristic image in which the first inspection value V1 or the second inspection value V2, and the position data or the movement direction data are displayed in association with each other; and an image output circuit 58 that outputs a processed image including the characteristic image generated by the characteristic image generation circuit 54. In response to this, the evaluation circuit 51 may evaluate the cognitive function based on the characteristic image generated by the characteristic image generation circuit 54. Here, the characteristic image is an image in which the result of a TMT inspection executed by a subject is visually represented so that an evaluator can easily evaluate the result, and the inspection values V1 and V2 and the position data or the movement direction data are displayed in association with each other.

Here, the sensor 14 may have any detection principle as long as it is possible to detect the contact of the contactor 80 on the display surface of the TMT inspection image I. Furthermore, the sensor 14 is provided on the display 18 in the present embodiment, but may be provided on the contactor 80 side.

In addition, in the present embodiment, the characteristic image generation circuit 54 includes an identification image generation circuit 56 that classifies the position data or the movement direction data into a plurality of groups based on the position or the direction on the coordinate plane, and generates the characteristic image in such a display form that inspection values corresponding to the respective groups are visually identifiable from one another.

The CPU 10 further includes: a passage detection circuit 32 that detects the passage of the contactor 80 at a passing point P based on coordinates of the contactor 80 acquired by the coordinate data acquisition circuit 42 from a detection signal of the sensor 14; and a memory 20 configured by, for example, a RAM and/or a ROM that stores at least the inspection data including the coordinate data and the time data acquired by the inspection data acquisition circuit 40 as well as the characteristic image generated by the characteristic image generation circuit 54. In this case, as described later, the passage detection circuit 32 sets, for each passing point P, a first coordinate region F1 for determining that the contactor 80 has passed through the passing point P and a second coordinate region F2 for determining that the contactor 80 stagnates at the passing point P (see FIG. 11), and detects the passage of the contactor 80 at the passing point P and the stagnation of the contactor 80 at the passing point P, and the movement of the contactor 80 from the passing point P. In addition, the passage detection circuit 32 includes a setting circuit 34 for variably setting the ranges of the first coordinate region F1 and the second coordinate region F2.

Note that the above-described various circuits whose operations are controlled by the control circuit 30 are illustrated as physically individual circuits in FIG. 2. However, a functional unit (or an apparatus) that integrates at least a part of or the entire of these circuits may be configured (for example, may be electronically put in a single package). In short, the circuits may exist in any form as long as the functions of these respective circuits are maintained.

Next, with reference to the flowcharts in FIG. 3 and FIGS. 4-21, a flow of a process of performing a TMT inspection using the terminal 1 (cognitive function evaluation system S) according to the present embodiment and evaluating/displaying the inspection result will be described.

First, a system user (hereinafter, simply referred to as a user) including a subject and an inspector such as a doctor can display the mode selection menu 19 on the display 18 by performing a predetermined input on, for example, a touch panel of the display 18 in the terminal 1. For example, in the mode selection menu 19, selection menus such as a TMT-A inspection, a TMT-B inspection, and the like are displayed for the user. This display is performed, for example, by the display circuit 17 under the control of the control circuit 30 based on an input signal from the display 18. Then, when the user selects an inspection form of the TMT inspection through the mode selection menu 19 (step S1), a TMT inspection image I corresponding to the selection is displayed on the display 18. Specifically, for example, when the user selects a TMT-A inspection on the mode selection menu 19, the inspection image generation circuit 25 electronically generates a TMT inspection image I for the TMT-A inspection obtained by setting the passing points at a plurality of positions on the coordinate plane based on the input signal from the mode selection menu 19 (display 18), and under the control of the control circuit 30, the display circuit 17 displays the TMT inspection image I for the TMT-A inspection as illustrated in FIG. 4(a) on the display 18 (inspection image generating and displaying step S2). In particular, in the present embodiment, after an introduction screen for the TMT-A inspection illustrated on an upper side of FIG. 4(a) is displayed, the TMT inspection image I for the TMT-A inspection illustrated on a lower side of FIG. 4(a) is displayed. As illustrated in the drawing, in the TMT inspection image I for the TMT-A inspection, circled numbers from 1 to 25 are randomly arranged as the passing points P based on a predetermined rule. At the time of executing the inspection, the user who is a subject moves the contactor 80 in a state of being in contact with the display surface of the TMT inspection image I to draw a line to connect the numbers in order from 1 to 25. Until the end of the inspection when the contactor 80 reaches 25, at least the time when the contactor 80 reaches each passing point P, the time when the contactor 80 starts moving from each passing point P, and the like are measured by the timer 16, and the movement direction between the passing points P, the positions of the passing points P, and the like are also recorded.

On the other hand, when the user selects a TMT-B inspection on the mode selection menu 19, the inspection image generation circuit 25 electronically generates a TMT inspection image I for the TMT-B inspection obtained by setting the passing points at a plurality of positions on the coordinate plane based on the input signal from the mode selection menu 19 (display 18), and under the control of the control circuit 30, the display circuit 17 displays the TMT inspection image I for the TMT-B inspection similar to that illustrated in FIG. 4(b) on the display 18. Also in this case, after an introduction screen for the TMT-B inspection illustrated on an upper side of FIG. 4(b) is displayed, the TMT inspection image I for the TMT-B inspection illustrated on a lower side of FIG. 4(b) is displayed. As illustrated in the drawing, in the TMT inspection image I for the TMT-B inspection, 13 numbers from 1 to 13 as well as 12 hiragana characters (a, i, u . . . sa, shi) are randomly arranged as the passing points P based on a predetermined rule. At the time of executing the inspection, the user who is a subject moves the contactor 80 in a state of being in contact with the display surface of the TMT inspection image I to draw a line to connect the numbers and the hiragana characters alternately in order. Until the end of the inspection when the contactor 80 reaches 13, at least the time when the contactor 80 reaches each passing point P, the time when the contactor 80 starts moving from each passing point P, and the like are measured by the timer 16, and the movement direction between the passing points P, the positions of the passing points P, and the like are also recorded.

Note that a TMT inspection image for a TMT-J inspection (not illustrated) includes several patterns having arrangement forms of numbers and alphabets or the like different from those in the inspection images I for the TMT-A inspection and the TMT-B inspection, and when the user selects the TMT-J inspection on the mode selection menu 19, the TMT inspection image for the TMT-J inspection having a predetermined pattern which is randomly selected by the control circuit 30 is displayed on the display 18, and the user can start the TMT-J inspection.

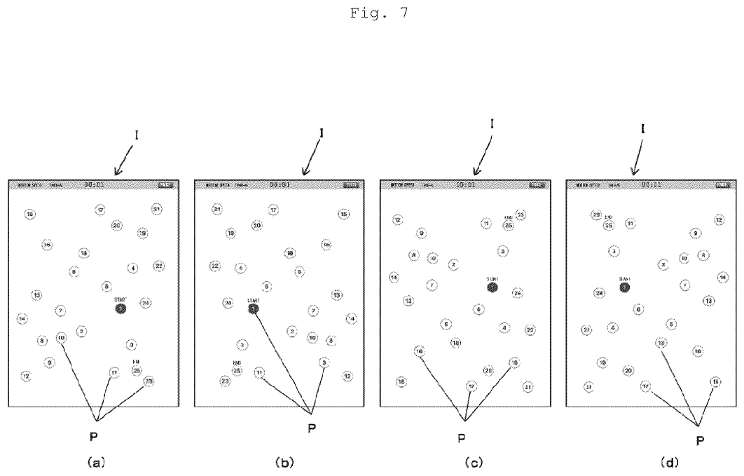

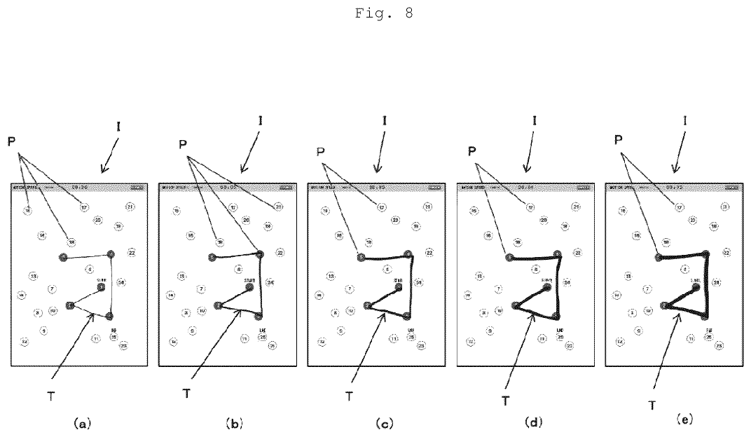

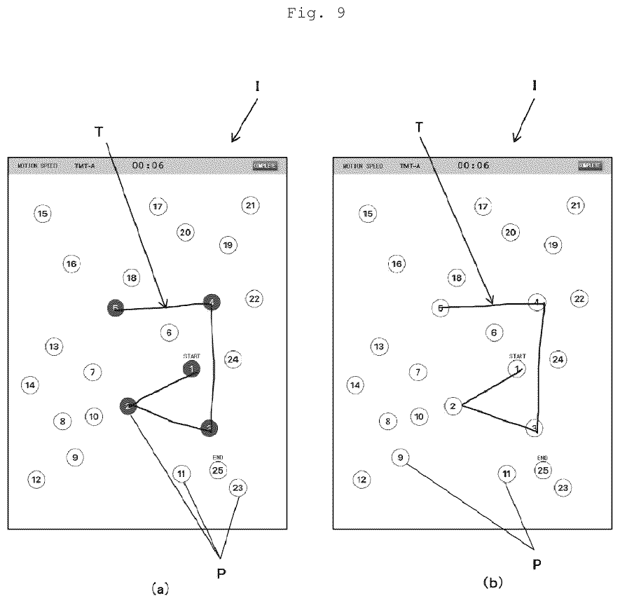

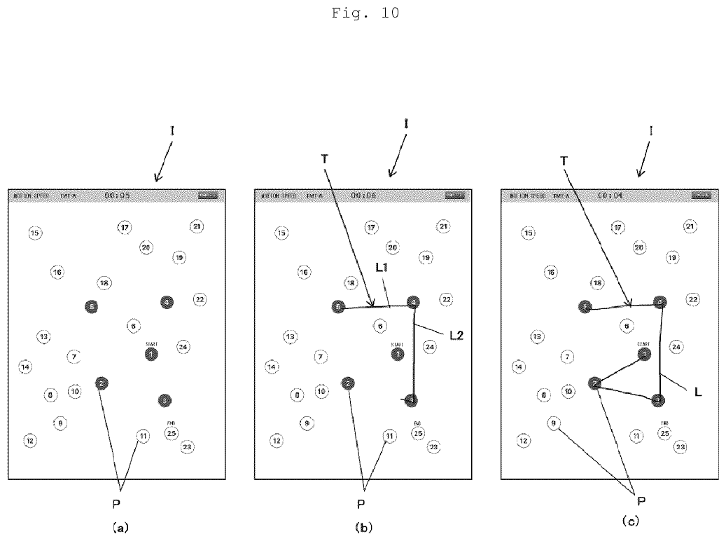

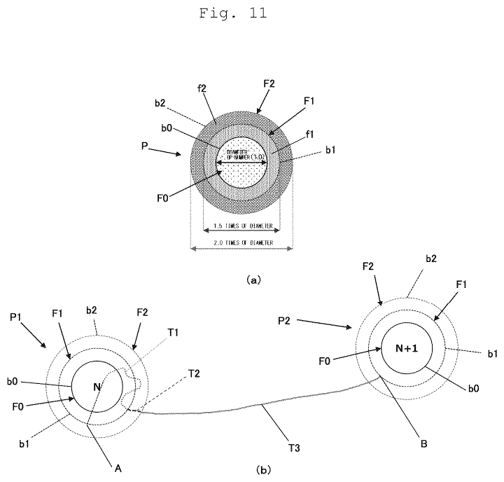

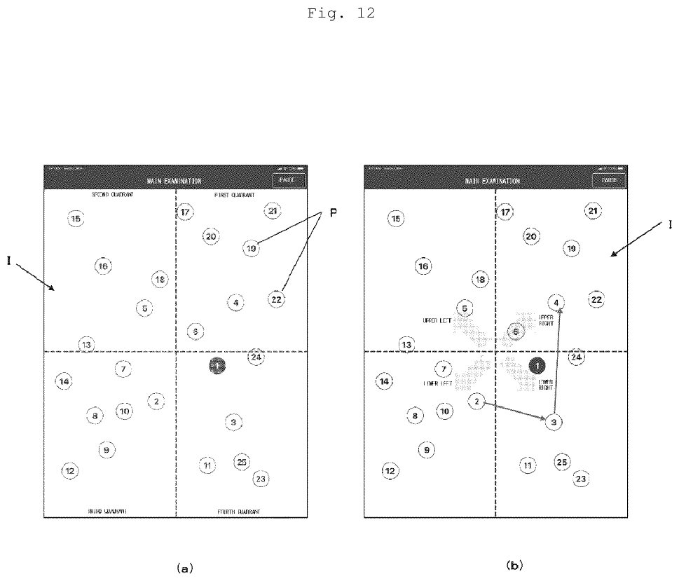

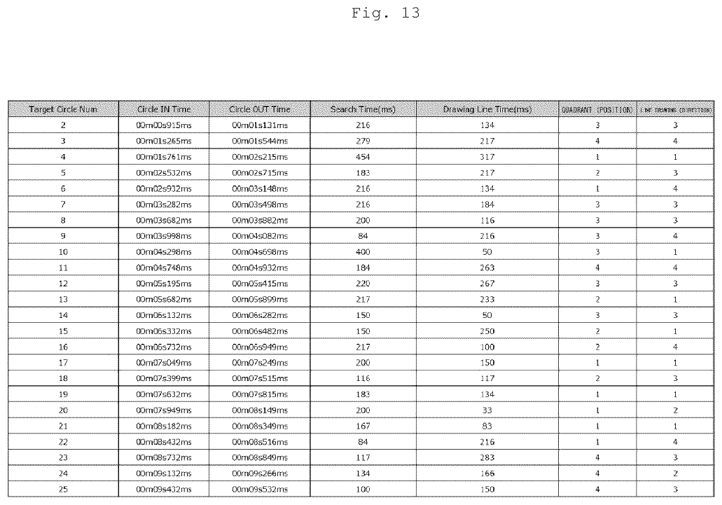

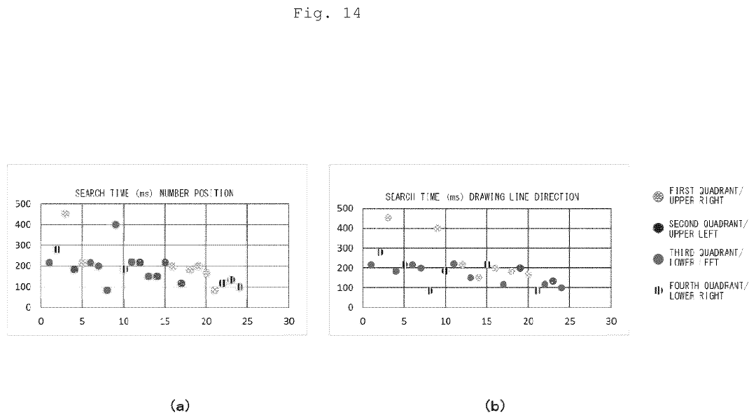

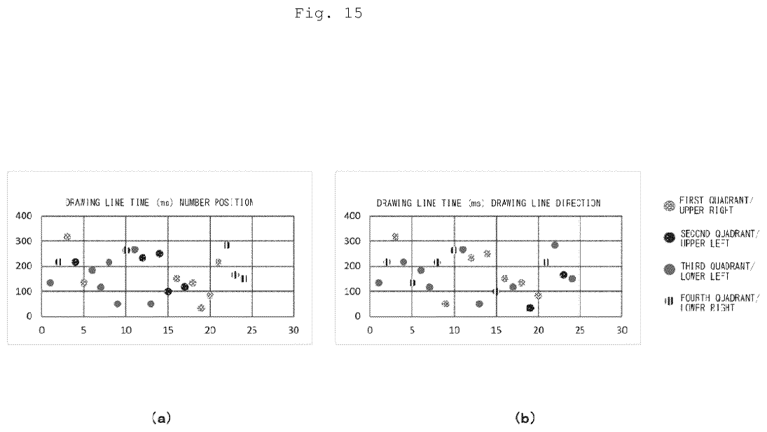

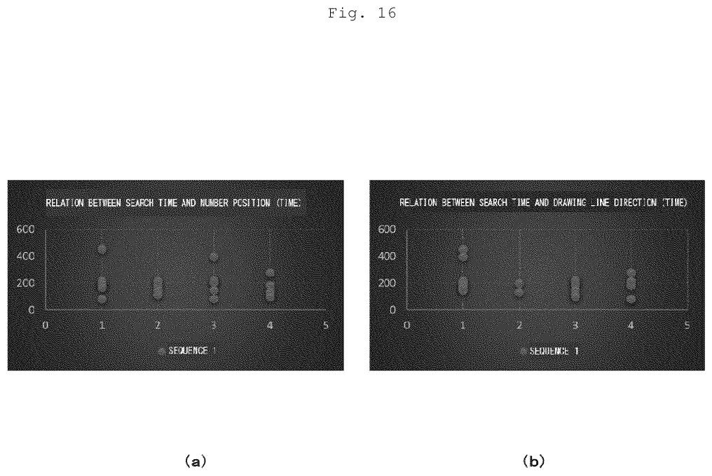

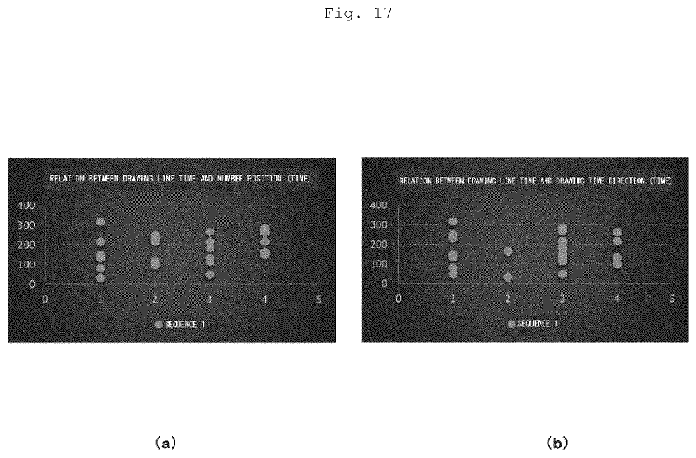

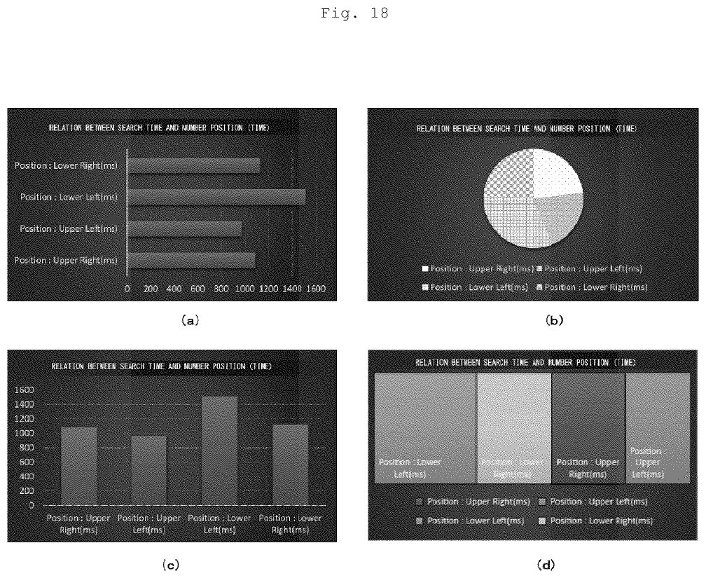

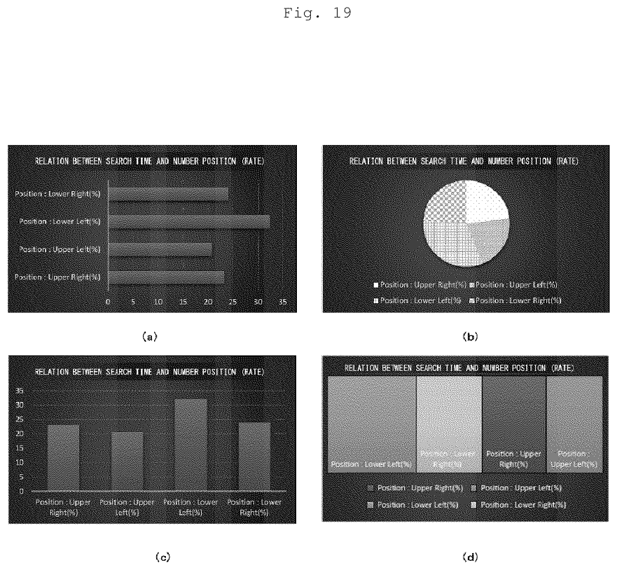

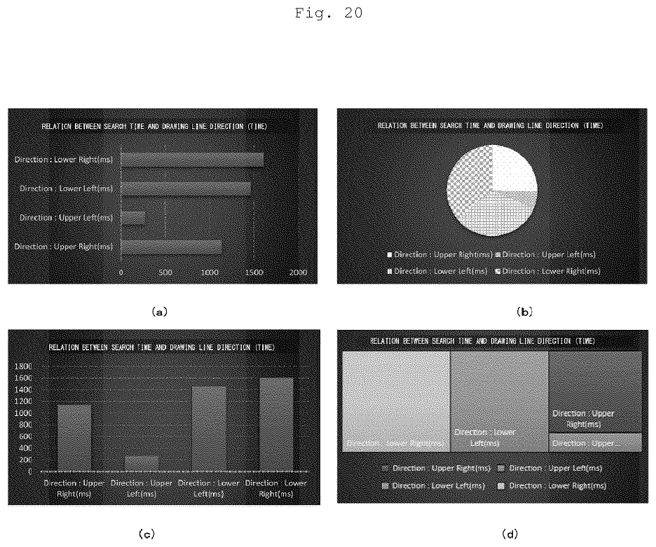

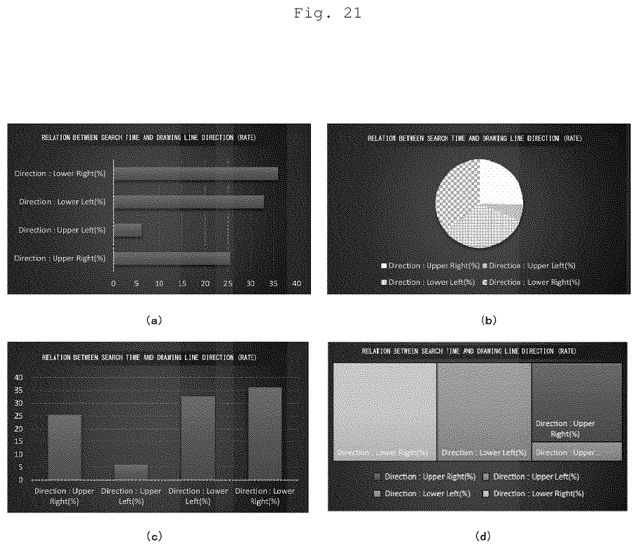

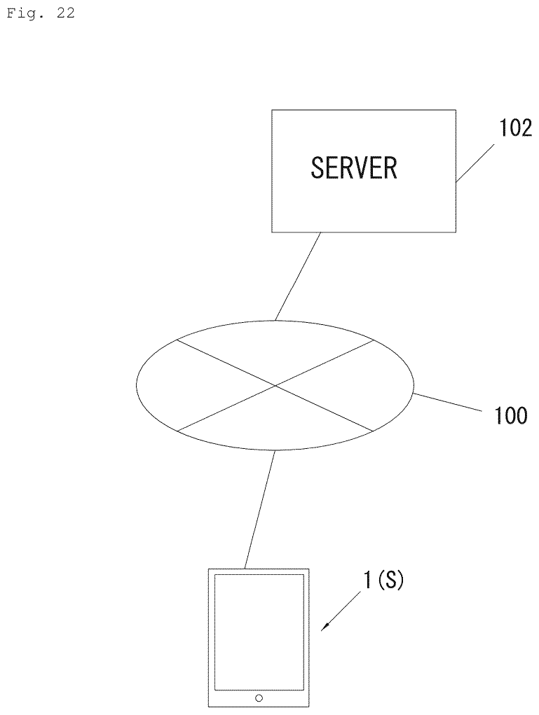

Furthermore, at the start of such a TMT inspection, the user can display a display setting screen for the TMT inspection as illustrated in FIG. 5 by performing a predetermined input on, for example, a touch panel of the display 18 in the terminal 1, and can perform various settings related to the display. Specifically, for example, when the direction of the vertical version is selected on the display setting screen in FIG. 5, a TMT inspection image I is displayed in the vertical direction on the display 18 as illustrated in FIG. 6(a) by the display circuit 17 under the control of the control circuit 30. On the other hand, when the direction of the horizontal version is selected on the display setting screen in FIG. 5, the TMT inspection image I is displayed in the horizontal direction as illustrated in FIG. 6(b). When a pattern A is selected on the display setting screen in FIG. 5, the inspection image generation circuit 25 and the display circuit 17 displays a predetermined TMT inspection image I as illustrated in FIG. 7(a) on the display 18 under the control of the control circuit 30. When a pattern B is selected on the display setting screen in FIG. 5, a TMT inspection image I obtained by flipping the image horizontally with respect to the pattern A as illustrated in FIG. 7(b) is displayed on the display 18. When a pattern C is selected on the display setting screen in FIG. 5, a TMT inspection image I obtained by flipping the image vertically with respect to the pattern A as illustrated in FIG. 7(c) is displayed on the display 18. When a pattern D is selected on the display setting screen in FIG. 5, a TMT inspection image I obtained by flipping the image vertically and horizontally with respect to the pattern A as illustrated in FIG. 7(d) is displayed on the display 18. Note that when random is selected on the display setting screen in FIG. 5, a TMT inspection image I having a pattern randomly determined by the control circuit 30 from among the patterns A to D is displayed on the display 18.

In the display setting screen in FIG. 5, the user can optionally set a display form such that a display line of the drawing trajectory drawn on a display surface of the TMT inspection image I is gradually thickened, for example, from stage 1 to stage 5 (for example, the display line may be set in milliunits). For example, when the thickness of the display line is set to the first stage on the display setting screen in FIG. 5, the display line of the drawing trajectory T as illustrated in FIG. 8(a) is displayed by the display circuit 17 on the TMT inspection image I under the control of the control circuit 30. When the thickness of the display line is set to the second stage on the display setting screen in FIG. 5, the display line of the drawing trajectory T as illustrated in FIG. 8(b) is displayed on the TMT inspection image I. When the thickness of the display line is set to the third stage on the display setting screen in FIG. 5, the display line of the drawing trajectory T as illustrated in FIG. 8(c) is displayed on the TMT inspection image I. When the thickness of the display line is set to the fourth stage on the display setting screen in FIG. 5, the display line of the drawing trajectory T as illustrated in FIG. 8(d) is displayed on the TMT inspection image I. When the thickness of the display line is set to the fifth stage on the display setting screen in FIG. 5, the display line of the drawing trajectory T as illustrated in FIG. 8(e) is displayed on the TMT inspection image I.

In addition, on the display setting screen in FIG. 5, it is possible to set (setting of color change at the time of touch) whether or not the color of the passing point P changes when the contactor 80 is coming into contact with the passing point P (passing through the passing point P). Specifically, when the setting is performed on the display setting screen in FIG. 5 such that, when the contactor 80 is passing through (coming into contact with) the passing point P, the color of the passing point P changes, the color of the passing point P passed by the contactor 80 drawing the drawing trajectory T on the TMT inspection image I is changed by the display circuit 17 under the control of the control circuit 30 as illustrated in FIG. 9(a) (in the drawing, circled numbers as the passing points P which has been changed in color is filled in black). On the other hand, when the setting is performed on the display setting screen in FIG. 5 such that, when the contactor 80 is passing through (coming into contact with) the passing point P, the color of the passing point P does not change, the color of the passing point P passed by the contactor 80 drawing the drawing trajectory T on the TMT inspection image I does not change as illustrated in FIG. 9(b).

In the display setting screen of FIG. 5, the display mode of the drawing trajectory by a user who is a subject is also settable. Specifically, in a case where the display mode of the drawing trajectory is set to “not displayed” (the drawing trajectory of the user is not visually displayed), even when the user moves the contactor 80 to draw the drawing trajectory, by the display circuit 17 under the control of the control circuit 30, the drawing trajectory is not displayed as a display line as illustrated in FIG. 10(a) (however, as illustrated in the drawing, the color of the communication point P through which the contactor 80 has already passed may change). When the display mode of the drawing trajectory is set to “point-to-point”, the drawing trajectory T of the user is visually displayed as a display line only in an area between the latest two passing points P (between a passing point P which has been lastly passed at the present time during the drawing and a passing point P which has been passed immediately therebefore) as illustrated in FIG. 10(b). For example, in this drawing, when the contactor 80 reaches a passing point P indicated by the number 5, a display line L2 connecting a passing point P indicated by the number 3 and a passage pattern P indicated by the number 4 disappears, and only a display line L1 connecting the passing point P indicated by the number 3 and the passage pattern P indicated by the number 4 remains (however, as illustrated in the drawing, the color of the communication point P through which the contactor 80 has already passed may change). Besides, when the display mode of the drawing trajectory is set to “fully displayed” (the entire of the drawing trajectory of the user is visually displayed), the entire of the drawing trajectory drawn chronologically by the user moving the contactor 80 is displayed as the display line L as illustrated in FIG. 10(c) (for example, as illustrated in the drawing, the color of the communication point P through which the contactor 80 has already passed also changes). In the display setting screen of FIG. 5, it is also possible to reset to the initial setting (default). In addition, the user may practice the TMT inspection on a practice screen which has been prepared in advance, before the start of the TMT inspection.

When a TMT test as described above is started by the user who is a subject (step S3), the inspection data acquisition circuit 40 acquires chronological data of a drawing trajectory drawn by the user moving the contactor 80 in a state of being in contact with a display surface of the TMT inspection image I so as to track the passing points P in a predetermined order (inspection data acquisition step S4). Specifically, in the inspection data acquisition step S4, the coordinate data acquisition circuit 42 acquires coordinate data corresponding to the position of the contactor 80 on the coordinate plane based on a detection signal from the sensor 14 that detects the contact of the contactor 80 with the display surface of the TMT inspection image I (coordinate data acquisition step), and the time data acquisition circuit 44 acquires time data associated with an acquisition time of each piece of the coordinate data by the timer 16. In this case, the coordinate data is also acquired as the position data related to the positions of the passing points P as well as the movement direction data related to the movement direction between the passing points P (movement direction directed from the first passing point P1 to the second passing point P2) and such data is acquired for all the passing points to be passed through.

Here, regarding the passage of the contactor 80 at a passing point P during the TMT inspection, as described above, the passage detection circuit 32 detects the passage of the contactor 80 at the passing point P based on the coordinates of the contactor 80 acquired by the coordinate data acquisition circuit 42 from the detection signal of the sensor 14 (passage detection step). In this case, the passage detection circuit 32 sets, for each passing point P, the first coordinate region F1 for determining that the contactor 80 has passed through the passing point P and the second coordinate region F2 for determining that the contactor 80 stagnates at the passing point P, and detects the passage of the contactor 80 at the passing point P and the stagnation of the contactor 80 at the passing point P, and the movement of the contactor 80 from the passing point P (a signal indicating the passage, the stagnation, and the movement detected in such a way is outputted from the passage detection circuit 32 to the control circuit 30). Specifically, for example, as shown in FIG. 11(a), at each passing point P, a circular reference region F0 defined by a circular boundary line b0 having a predetermined diameter with corresponding numbers, hiragana, alphabets displayed thereinside, a circular first coordinate region F1 defined inside by a circular boundary line b1 that is concentric with the reference region F0 and has a diameter that is, for example, 1.5 times the diameter of the reference region F0 ([circular reference region F0]+[annular region f1 defined by the boundary line b0 and the boundary line b1]), and a circular second coordinate region F2 defined inside by a circular boundary line b2 that is concentric with the reference region F0 and has a diameter that is, for example, twice the diameter of the reference region F0 ([circular reference region F0]+[annular region f1 defined by the boundary line b0 and the boundary line b1]+[annular region f2 defined by the boundary line b1 and the boundary line b2]). Then, as illustrated in FIG. 11(b), when an arbitrary one first passing point (accompanied by corresponding number N, for example) P1 among a plurality of passing points P on the TMT inspection image I and a second passing point (accompanied by the corresponding number N+1, for example) P2 to pass through next are taken as examples, the passage detection circuit 32 detects a time point at which the contactor 80 enters the first coordinate region F1 at the first passing point P1 (when the contactor 80 reaches a point A on the boundary line b1) as the contactor 80's passage at the first passing point P1; detects the contactor 80's movement (for example, the movement tracking the drawing trajectory indicated by broken lines T1 and T2 in FIG. 11(b)) in the second coordinate region F2 at the first passing point P1 as the contactor 80's stagnation at the first passing point P1, as well as detects the contactor 80's movement (the movement beyond the boundary line b2) from the inside of the second coordinate region F2 to the outside of the second coordinate region F2 at the first passing point P1 as the contactor 80's movement from the first passing point P1; and detects a time point at which the contactor 80 enters the first coordinate region F1 at the second passing point P2 (when the contactor 80 reaches a point B on the boundary line b1 at the second passing point P2) as the contactor 80's passage at the second passing point P2. Therefore, a drawing trajectory indicated by a solid line T3 in FIG. 11(b) corresponds to the contactor 80's point-to-point movement trajectory between the first passing point P1 and the second passing point P2, and a period for tracking the point-to-point movement trajectory corresponds to the time of contactor 80's movement between the first passing point P1 and the second passing point P2, which is computed by the computation circuit 52 as described later. In particular, in the present embodiment, the computation circuit 52 computes the movement time (second inspection value) by regarding a period of a drawing trajectory (a period of the drawing trajectory indicated by a thick broken line T2 in FIG. 11(b)) continuous with the point-to-point movement trajectory that exceeds the boundary line b1 and reaches the boundary line b2 as the movement period, too. Therefore, in response to this, in the present embodiment, a drawing trajectory indicated by the thin broken line T1 in FIG. 11(b) is regarded as a stagnation trajectory of the contactor 80 at the first passing point P1, and a period for tracking the stagnation trajectory is computed by the computation circuit 52 as a residence time (first inspection value) of the contractor 80 at the first passing point P1 as described later. However, a way of setting the coordinate region as well as a way of obtaining the residence time and the movement time are not limited thereto (for example, only a period of the drawing trajectory indicated by a solid line in FIG. 11(b) may be regarded as the movement time of the contactor 80 between the first passing point P1 and the second passing point P2.). For example, the ranges of the first coordinate region F1 and the second coordinate region F2 can be freely set (setting step) by the setting circuit 34, whereby it is possible to give free rein to the determination of passage, stagnation, and movement (an inspection having a degree of freedom depending on situations is enabled).

Once the TMT inspection executed by the user is ended while various types of inspection data are acquired as described above (step S5) (alternatively, in parallel with the TMT inspection), the data processing circuit 50 processes data acquired by the inspection data acquisition circuit 40 so as to make a result of the processing displayable on the display 18 as the inspection result (data processing and displaying step). Specifically, based on the coordinate data and the time data in the contactor 80 and the signal indicating the stagnation and the movement described above from the passage detection circuit 32, the computation circuit 52 in the data processing circuit 50 computes the first inspection value related to a residence time during which the contactor 80 stagnates at the first passing point P1 that is any one of the plurality of passing points from when it reaches the first passing point P1 until when it starts moving to a second passing point P2 to be passed through next, and the second inspection value related to a movement time required from when the contactor 80 starts moving from the first passing point P1 until when the contactor 80 reaches the second passing point P2, for all of the passing points P to be passed through (computation step S6), and the characteristic image generation circuit 54 in the data processing circuit 50 generates a characteristic image in which the first inspection value or the second inspection value computed by the computation circuit 52 and the position data or the movement direction data obtained from the coordinate data acquired by the inspection data acquisition circuit 40 are displayed in association with each other (characteristic image generation step S7).

The position data and the movement direction data obtained from the first inspection value and the second inspection value computed by the computation circuit 52 and the coordinate data acquired by the inspection data acquisition circuit 40 may be displayed on the display 18 via the image output circuit 58 as a processed image in the form of a table as illustrated in FIG. 13, for example. Here, the table in FIG. 13 is illustrated as data in the TMT inspection image I obtained by setting the passing points P at a plurality of positions on the coordinate plane as illustrated in FIG. 12, for example. In FIG. 13, “Target Circle Num” indicates the number of a target passing point P to be passed through next, and for example, when “Target Circle Num” is “4”, it indicates the movement from the passing point P with the number “3” to the passing point P with the number “4”. In addition, in FIG. 13, “Circle IN Time” indicates the time of reaching a corresponding passing point P (time of entering a first coordinate region F1 of a passing point P corresponding to “Target Circle Num”), and “Circle OUT Time” indicates the time of starting to move from the corresponding passing point P (in the present embodiment in which the period of a drawing trajectory continuous with a point-to-point movement trajectory that moves across the boundary line b1 and reaches the boundary line b2 (the period of a drawing trajectory indicated by the thick broken line T2 in FIG. 11(b)) is also regarded as the movement period, the time when the boundary line b1 of the passing point P corresponding to “Target Circle Num” is passed over in accordance with the movement). Furthermore, in FIG. 13, “Search Time” indicates the residence time at a corresponding passing point P, and “Drawing Line Time” indicates the movement time to the corresponding passing point P, that is, for example, when “Target Circle Num” is “4”, the time associated with the movement from the passing point P with the number “3” to the passing point P with the number “4”. Furthermore, in FIG. 13, “quadrant (position)” indicates the position of a corresponding passing point P on the coordinate plane, specifically, in a case where the coordinate plane is divided vertically and horizontally into four regions as illustrated in FIG. 12(a), that is, a first quadrant, a second quadrant, a third quadrant, and a fourth quadrant, a quadrant in which the corresponding passing point is located is indicated by the number of the quadrant (for example, since the passing point P accompanied by the number “2” is located in the third quadrant in FIG. 12, in a case where “Target Circle Num” is “2” in the table in FIG. 13, “quadrant (position)” therefor is indicated by the number “3”). Furthermore, in FIG. 13, “line drawing (direction)” indicates the movement direction to a corresponding passing point P, that is, for example, when “Target Circle Num” is “4”, the movement direction from the passing point P with the number “3” to the passing point P with the number “4”. In particular, here, as indicated by the arrows in FIG. 12(b), the movement direction is divided into four directions: a direction toward “upper right”, a direction toward “upper left”, a direction toward “lower left”, and a direction toward “lower right”. The direction toward “upper right” is indicated by the number “1”, the direction toward “upper left” is indicated by the number “2”, the direction toward “lower left” is indicated by the number “3”, and the direction toward “lower right” is indicated by the number “4”. Therefore, in the table in FIG. 13, for example, when “Target Circle Num” is “3”, the movement direction is the “lower right” direction from the passing point P with the number “2” to the passing point P with the number “3”, and thus, the “drawing line (direction)” therefor is indicated by the number “4”. When “Target Circle Num” is “4”, the movement direction is the “upper right” direction from the passing point P with the number “3” to the passing point P with the number “4”, and thus, the “drawing line (direction)” therefor is indicated by the number “1”.

FIGS. 14-21 illustrate examples of characteristic images generated by the characteristic image generation circuit 54. A characteristic image illustrated in FIG. 14(a) is an image in which the residence time (search time of searching for the passing point P to be passed through next: millisecond) that is the first inspection value computed by the computation circuit 52, and the position of the passing point P obtained from the coordinate data acquired by the inspection data acquisition circuit 40 are displayed in association with each other for all the passing points (passing points with the numbers 1-25) in relation to the data illustrated in FIG. 13, and the horizontal axis indicates the numbers respectively associated with 25 passing points and the vertical axis indicates the residence time. In particular, this characteristic image is generated by the identification image generation circuit 56 as a characteristic image in such a display form that the position data is divided into a plurality of groups based on the position on the coordinate plane, that is, a group of the first quadrant, a group of the second quadrant, a group of the third quadrant, and a group of the fourth quadrant, and the first inspection values (residence time) corresponding to the respective groups are visually identifiable from one another (identification image generation step). Here, as the “display form that . . . are visually identifiable”, the data (first inspection values) corresponding to the respective groups are displayed so that the data can be visually distinguished from one another at a glance in accordance with the difference in color or pattern (displayed with colored or patterned dots). However, the data (inspection values) corresponding to the respective groups may be visually distinguishable from one another at a glance in accordance with the other differences. In addition, such grouping can be selected by a user through the mode selection menu 19, and the control circuit 80 controls the characteristic image generation circuit 54 (identification image generation circuit 56) based on an input signal from the mode selection menu associated with the selection to generate an identifiably displayed image.

Besides, a characteristic image illustrated in FIG. 14(b) is an image in which the residence time (search time of searching for the passing point P to be passed through next: millisecond) that is the first inspection value computed by the computation circuit 52, and the movement direction between the passing points P obtained from the coordinate data acquired by the inspection data acquisition circuit 40 are displayed in association with each other for all the passing points (passing points with the numbers 1-25) in relation to the data illustrated in FIG. 13, and the horizontal axis indicates the numbers respectively associated with 25 passing points and the vertical axis indicates the residence time. In particular, this characteristic image is generated by the identification image generation circuit 56 as a characteristic image in such a display form that the movement direction data is divided into a plurality of groups based on the direction on the coordinate plane, that is, a group of the movement direction toward “upper right”, a group of the movement direction toward “upper left”, a group of the movement direction toward “lower left”, and a group of the movement direction toward “lower right”, and the first inspection values (residence time) corresponding to the respective groups are visually identifiable from one another (identification image generation step). Again, as the “display form that . . . are visually identifiable”, the data (first inspection values) corresponding to the respective groups are displayed so that the data can be visually distinguished from one another at a glance in accordance with the difference in color or pattern (displayed with colored or patterned dots). However, the data (inspection values) corresponding to the respective groups may be visually distinguishable from one another at a glance in accordance with the other differences.

A characteristic image illustrated in FIG. 15(a) is an image in which the movement time (drawing line time of drawing a line toward the passing point P to be passed through next: millisecond) that is the second inspection value computed by the computation circuit 52, and the position of the passing point P obtained from the coordinate data acquired by the inspection data acquisition circuit 40 are displayed in association with each other for all the passing points (passing points with the numbers 1-25) in relation to the data illustrated in FIG. 13, and the horizontal axis indicates the numbers respectively associated with 25 passing points and the vertical axis indicates the movement time. In particular, this characteristic image is generated by the identification image generation circuit 56 as a characteristic image in such a display form that the position data is divided into a plurality of groups based on the position on the coordinate plane, that is, a group of the first quadrant, a group of the second quadrant, a group of the third quadrant, and a group of the fourth quadrant, and the second inspection values (movement time) corresponding to the respective groups are visually identifiable from one another (identification image generation step). Again, as the “display form that . . . are visually identifiable”, the data (first inspection values) corresponding to the respective groups are displayed so that the data can be visually distinguished from one another at a glance in accordance with the difference in color or pattern (displayed with colored or patterned dots). However, the data (inspection values) corresponding to the respective groups may be visually distinguishable from one another at a glance in accordance with the other differences.

Besides, a characteristic image illustrated in FIG. 15(b) is an image in which the movement time (drawing line time of drawing a line toward the passing point P to be passed through next: millisecond) that is the second inspection value computed by the computation circuit 52, and the movement direction between the passing points P obtained from the coordinate data acquired by the inspection data acquisition circuit 40 are displayed in association with each other for all the passing points (passing points with the numbers 1-25) in relation to the data illustrated in FIG. 13, and the horizontal axis indicates the numbers respectively associated with 25 passing points and the vertical axis indicates the movement time. In particular, this characteristic image is generated by the identification image generation circuit 56 as a characteristic image in such a display form that the movement direction data is divided into a plurality of groups based on the direction on the coordinate plane, that is, a group of the movement direction toward “upper right”, a group of the movement direction toward “upper left”, a group of the movement direction toward “lower left”, and a group of the movement direction toward “lower right”, and the second inspection values (movement time) corresponding to the respective groups are visually identifiable from one another (identification image generation step). Again, as the “display form that . . . are visually identifiable”, the data (first inspection values) corresponding to the respective groups are displayed so that the data can be visually distinguished from one another at a glance in accordance with the difference in color or pattern (displayed with colored or patterned dots). However, the data (inspection values) corresponding to the respective groups may be visually distinguishable from one another at a glance in accordance with the other differences.

A characteristic image illustrated in FIG. 16(a) is an image (data at the respective passing points P are represented in dots) in which the residence time (search time of searching for the passing point P to be passed through next: millisecond) that is the first inspection value computed by the computation circuit 52, and the position of the passing point P obtained from the coordinate data acquired by the inspection data acquisition circuit 40 are displayed in association with each other for all the passing points in relation to the data illustrated in FIG. 13, and the horizontal axis indicates the quadrant position on the coordinate plane and the vertical axis indicates the residence time. That is, this characteristic image is generated by the identification image generation circuit 56 as a characteristic image in such a display form that the position data is divided into a plurality of groups based on the position on the coordinate plane, that is, a group of the first quadrant, a group of the second quadrant, a group of the third quadrant, and a group of the fourth quadrant, and the first inspection values (residence time) corresponding to the respective groups are visually identifiable from one another.

A characteristic image illustrated in FIG. 16(b) is an image (data at the respective passing points P are represented in dots) in which the residence time (search time of searching for the passing point P to be passed through next: millisecond) that is the first inspection value computed by the computation circuit 52, and the movement direction between the passing point P obtained from the coordinate data acquired by the inspection data acquisition circuit 40 are displayed in association with each other for all the passing points in relation to the data illustrated in FIG. 13, and the horizontal axis indicates the movement direction on the coordinate plane (the direction toward “upper right” is indicated by the number “1”, the direction toward “upper left” is indicated by the number “2”, the direction toward “lower left” is indicated by the number “3”, and the direction toward “lower right” is indicated by the number “4”) and the vertical axis indicates the residence time. That is, this characteristic image is generated by the identification image generation circuit 56 as a characteristic image in such a display form that the movement direction data is divided into a plurality of groups based on the direction on the coordinate plane, that is, a group of the movement direction toward “upper right”, a group of the movement direction toward “upper left”, a group of the movement direction toward “lower left”, and a group of the movement direction toward “lower right”, and the first inspection values (residence time) corresponding to the respective groups are visually identifiable from one another.

A characteristic image illustrated in FIG. 17(a) is an image (data at the respective passing points P are represented in dots) in which the movement time (drawing line time of drawing a line toward the passing point P to be passed through next: millisecond) that is the second inspection value computed by the computation circuit 52, and the position of the passing point P obtained from the coordinate data acquired by the inspection data acquisition circuit 40 are displayed in association with each other for all the passing points in relation to the data illustrated in FIG. 13, and the horizontal axis indicates the quadrant position on the coordinate plane and the vertical axis indicates the movement time. That is, this characteristic image is generated by the identification image generation circuit 56 as a characteristic image in such a display form that the position data is divided into a plurality of groups based on the position on the coordinate plane, that is, a group of the first quadrant, a group of the second quadrant, a group of the third quadrant, and a group of the fourth quadrant, and the second inspection values (movement time) corresponding to the respective groups are visually identifiable from one another.

A characteristic image illustrated in FIG. 17(b) is an image (data at the respective passing points P are represented in dots) in which the movement time (drawing line time of drawing a line toward the passing point P to be passed through next: millisecond) that is the second inspection value computed by the computation circuit 52, and the movement direction between the passing point P obtained from the coordinate data acquired by the inspection data acquisition circuit 40 are displayed in association with each other for all the passing points in relation to the data illustrated in FIG. 13, and the horizontal axis indicates the movement direction on the coordinate plane (the direction toward “upper right” is indicated by the number “1”, the direction toward “upper left” is indicated by the number “2”, the direction toward “lower left” is indicated by the number “3”, and the direction toward “lower right” is indicated by the number “4”) and the vertical axis indicates the movement time. That is, this characteristic image is generated by the identification image generation circuit 56 as a characteristic image in such a display form that the movement direction data is divided into a plurality of groups based on the direction on the coordinate plane, that is, a group of the movement direction toward “upper right”, a group of the movement direction toward “upper left”, a group of the movement direction toward “lower left”, and a group of the movement direction toward “lower right”, and the second inspection values (movement time) corresponding to the respective groups are visually identifiable from one another.

Each characteristic image illustrated in FIG. 18 is an identification image in which the first inspection value related to the residence time (search time: millisecond) computed by the computation circuit 52 and the position of the passing point obtained from the coordinate data acquired by the inspection data acquisition circuit 40 are displayed in association with each other in relation to the data illustrated in FIG. 13. This characteristic image is an identification image represented in such a display form that the position data is divided into a plurality of groups based on the position on the coordinate plane, that is, a group of the first quadrant (“Upper Right”), a group of the second quadrant (“Upper Left”), a group of the third quadrant (“Lower Left”), and a group of the fourth quadrant (“Lower Right”), and the first inspection values corresponding to the respective groups are visually identifiable from one another. Specifically, each characteristic image illustrated in FIG. 18 is obtained by the computation circuit 52 computing the sums of the residence times at the respective passing points P for the respective groups, and by the identification image generation circuit 56 generating an identification image such that the sums are displayed for the respective groups. That is, in the characteristic image in FIG. 18(a), the sums of the residence times for the respective quadrants are displayed in an identifiable manner using a bar graph, the horizontal axis indicates the sums of the residence times and the vertical axis indicates each quadrant. In the characteristic image in FIG. 18(b), the sums of the residence times for the respective quadrants are displayed in different colors or different patterns in a circular graph. On the other hand, in the characteristic image in FIG. 18(c), the sums of the residence times for the respective quadrants are displayed in an identifiable manner using a bar graph, the horizontal axis indicates each quadrant and the vertical axis indicates the sums of the residence times. In the characteristic image in FIG. 18(d), the sums of the residence times for the respective quadrants are displayed in different colors or different patterns in a band graph.

Each characteristic image illustrated in FIG. 19 is an identification image in which the first inspection value related to the residence time (search time: millisecond) computed by the computation circuit 52 and the position of the passing point obtained from the coordinate data acquired by the inspection data acquisition circuit 40 are displayed in association with each other in relation to the data illustrated in FIG. 13. Similarly to the case of FIG. 18, this characteristic image is an identification image represented in such a display form that the position data is divided into a plurality of groups based on the position on the coordinate plane, that is, a group of the first quadrant (“Upper Right”), a group of the second quadrant (“Upper Left”), a group of the third quadrant (“Lower Left”), and a group of the fourth quadrant (“Lower Right”), and the first inspection values corresponding to the respective groups are visually identifiable from one another. Specifically, each characteristic image illustrated in FIG. 19 is obtained by the computation circuit 52 computing the sums of the residence times at the respective passing points P for the respective groups and computing the rates of the sums of the respective groups with respect to the sum of all groups, and by the identification image generation circuit 56 generating an identification image such that the rates are displayed for the respective groups. That is, in the characteristic image in FIG. 19(a), the rates (%) of the residence times for the respective quadrants are displayed in an identifiable manner using a bar graph, the horizontal axis indicates the rates of the residence times and the vertical axis indicates each quadrant. In the characteristic image in FIG. 19(b), the rates of the residence times for the respective quadrants are displayed in different colors or different patterns in a circular graph. On the other hand, in the characteristic image in FIG. 19(c), the rates of the residence times for the respective quadrants are displayed in an identifiable manner using a bar graph, the horizontal axis indicates each quadrant and the vertical axis indicates the rates of the residence times. In the characteristic image in FIG. 19(d), the rates of the residence times for the respective quadrants are displayed in different colors or different patterns in a band graph.

Each characteristic image illustrated in FIG. 20 is an identification image in which the first inspection value related to the residence time (search time: millisecond) computed by the computation circuit 52 and the movement direction (drawing line direction) between the passing points obtained from the coordinate data acquired by the inspection data acquisition circuit 40 are displayed in association with each other in relation to the data illustrated in FIG. 13. This characteristic image is an identification image represented in such a display form that the movement direction data is divided into a plurality of groups based on the direction on the coordinate plane, that is, a group of the movement direction toward “Upper Right”, a group of the movement direction toward “Upper Left”, a group of the movement direction toward “Lower Left”, and a group of the movement direction toward “Lower Right”, and the first inspection values corresponding to the respective groups are visually identifiable from one another. Specifically, each characteristic image illustrated in FIG. 20 is obtained by the computation circuit 52 computing the sums of the residence times at the respective passing points P for the respective groups, and by the identification image generation circuit 56 generating an identification image such that the sums are displayed for the respective groups. That is, in the characteristic image in FIG. 20(a), the sums of the residence times for the respective movement directions are displayed in an identifiable manner using a bar graph, the horizontal axis indicates the sums of the residence times and the vertical axis indicates each movement direction. In the characteristic image in FIG. 20(b), the sums of the residence times for the respective movement directions are displayed in different colors or different patterns in a circular graph. On the other hand, in the characteristic image in FIG. 20(c), the sums of the residence times for the respective movement directions are displayed in an identifiable manner using a bar graph, the horizontal axis indicates each movement direction and the vertical axis indicates the sums of the residence times. In the characteristic image in FIG. 20(d), the sums of the residence times for the respective movement directions are displayed in different colors or different patterns in a band graph.

Each characteristic image illustrated in FIG. 21 is an identification image in which the first inspection value related to the residence time (search time: millisecond) computed by the computation circuit 52 and the movement direction (drawing line direction) between the passing points obtained from the coordinate data acquired by the inspection data acquisition circuit 40 are displayed in association with each other in relation to the data illustrated in FIG. 13. Similarly to the case of FIG. 20, this characteristic image is an identification image represented in such a display form that the movement direction data is divided into a plurality of groups based on the direction on the coordinate plane, that is, a group of the movement direction toward “Upper Right”, a group of the movement direction toward “Upper Left”, a group of the movement direction toward “Lower Left”, and a group of the movement direction toward “Lower Right”, and the first inspection values corresponding to the respective groups are visually identifiable from one another. Specifically, each characteristic image illustrated in FIG. 21 is obtained by the computation circuit 52 computing the sums of the residence times at the respective passing points P for the respective groups and computing the rates of the sums of the respective groups with respect to the sum of all groups, and by the identification image generation circuit 56 generating an identification image such that the rates are displayed for the respective groups. That is, in the characteristic image in FIG. 21(a), the rates (%) of the residence times for the respective movement directions are displayed in an identifiable manner using a bar graph, the horizontal axis indicates the rates of the residence times and the vertical axis indicates each movement direction. In the characteristic image in FIG. 21(b), the rates of the residence times for the respective movement directions are displayed in different colors or different patterns in a circular graph. On the other hand, in the characteristic image in FIG. 21(c), the rates of the residence times for the respective movement directions are displayed in an identifiable manner using a bar graph, the horizontal axis indicates each movement direction and the vertical axis indicates the rates of the residence times. In the characteristic image in FIG. 21(d), the rates of the residence times for the respective movement directions are displayed in different colors or different patterns in a band graph.

The first inspection value and the second inspection value obtained through the computation by the computation circuit 52, the position data and the movement direction data obtained from the coordinate data acquired by the inspection data acquisition circuit 40, and the characteristic image generated by the characteristic image generation circuit 54 are used for a cognitive function evaluation executed by the evaluation circuit 51. That is, the evaluation circuit 61 evaluates the cognitive function of a user who is a subject who took a TMT inspection, based on the first inspection value and the second inspection value, or based on the first inspection value, the second inspection value, the position data, and the movement direction data, or based on the characteristic image, or based on any combination thereof (evaluation step S8). In this case, a cognitive function evaluation executed by the evaluation circuit 51 is performed by, for example, the method described at the beginning of the present embodiment. The evaluation circuit 51 may also estimate an MMSE score based on the first and second inspection values, the position data, and the movement direction data, or any combination thereof. Also in this case, the estimation is performed by, for example, the method described at the beginning of the present embodiment.

The inspection result including the result of evaluation executed by the evaluation circuit 51 is outputted by the inspection result outputting circuit 53 and displayed on the display 18 (inspection result outputting and displaying step S9). The characteristic image generated by the characteristic image generation circuit 54 is outputted by the image output circuit 58 and displayed on the display 18 (image outputting and displaying step).

The processed image including the characteristic image, the inspection result including the evaluation result, and the inspection data including the coordinate data and the time data acquired by the inspection data acquisition circuit 40 are stored in the memory 20 (data storage step S10). By storing the inspection data and the like in the memory 20 in this manner, the data can be accumulated in the memory and required data can be read out in a timely manner as necessary. In addition, for example, it becomes possible to evaluate the progress of symptoms by comparing pieces of the accumulated history data, or execute a final authorization of the evaluation based on the data accumulated in the memory.

Furthermore, as illustrated in FIG. 22 described above, the terminal 1 having the above-described configuration may also take a usage form of being connected to the server 102 via the communication means (network) 100. In this case, the communication means 100 exchanges information between the terminal 1 and the server 102 and may establish either wired communication or wireless communication. Examples of the communication means 100 include a line using a wired cable, a wired telephone line, a mobile telephone line, a WiFi line, and the like.

Furthermore, in such a usage form, the terminal 1 also has a communication circuit 12 (see FIG. 2) that enables transmission and reception of data to and from the server 102 via the communication means 100. Accordingly, for example, the terminal 1 can send the inspection data obtained by itself to the server 102 and executes the cognitive function evaluation and the like thereon on the server 102 side. On the other hand, the terminal 1 can also change or add various functions based on the information from the server 102 (for example, an analysis function can be updated (an analysis program is downloadable on the tablet (terminal 1)) . . . a more detailed analysis function is provided on the server 102 side and the analysis result in the server 102 is transmitted to the tablet). Besides, a database in the server 102 may store therein a subject's user ID, the subject's personal data such as age, address, and sex, an inspection execution date, an inspection result, and the like (main function). In addition, it is preferable that any medical institution can access the server 102. Furthermore, it is preferable that the database can be utilized as big data related to dementia.

As described above, according to the terminal 1 (cognitive function evaluation system S) of the present embodiment, the first inspection value data related to the “residence time” and the second inspection value data related to the “movement time” are extracted from the inspection data obtained by executing the inspection based on the TMT for all the passing points to be passed through, and the subject's cognitive function is evaluated based on the first inspection value data and the second inspection value data which have been extracted. Accordingly, the correlation between the TMT and the MMSE increases, and the number of times of TMT execution required for estimating the MMSE score decreases, whereby a highly accurate cognitive function evaluation can be performed in a short time. Besides, in addition to the first and second inspection value data, the position data related to the “position” of the passing point and the movement direction data related to the “movement direction” between the passing points are extracted for all the passing points to be passed through, and the subject's cognitive function can be evaluated based on the first inspection value data, the second inspection value data, the position data, and the movement direction data which have been extracted. Therefore, it becomes possible to execute, in a shorter time, a highly accurate cognitive function evaluation in which the correlation between the TMT and the MMSE further increases.

Note that the present invention is not limited to the above-described embodiment, and various modifications can be made without departing from the gist thereof. For example, in the present invention, another process may be further added between the above-described processing steps, and the order of the steps may be partially changed. In addition, in the above-described embodiment, the TMT inspection and the evaluation thereof are automated by the terminal. However, it is needless to say that the present invention can be applied to any action of performing the cognitive function evaluation and the MMSE score estimation using a residence time and the like without automating the whole. Furthermore, the whole or a part of the above-described embodiment may be combined without departing from the gist of the present invention, or a part of the configuration may be omitted from one of the above-described embodiments.

### REFERENCE SIGNS LIST

1 terminal
14 sensor
16 timer
18 display
19 mode selection menu
20 memory
25 inspection image generation circuit
30 control circuit
32 passage detection circuit
34 setting circuit
40 inspection data acquisition circuit
42 coordinate data acquisition circuit
44 time data acquisition circuit
50 data processing circuit
51 evaluation circuit
52 computation circuit
53 inspection result outputting circuit
54 characteristic image generation circuit
56 identification image generation circuit
58 image output circuit
80 contactor
S cognitive function evaluation system

1. A cognitive function evaluation method comprising:
a TMT execution step for executing an inspection based on a TMT for urging a subject to connect a plurality of passing points by a line such that the line passes through the passing points sequentially based on a predetermined rule;
a data extraction step for extracting, from inspection data obtained through the TMT execution step, first inspection value data related to a residence time during which a drawn line drawn by the subject sequentially tracking the passing points stagnates at a first passing point that is any one of the plurality of passing points from when the drawn line reaches the first passing point until when the drawn line starts moving to a second passing point to be passed through next, and second inspection value data related to a movement time required from when the drawn line starts moving from the first passing point until when the drawn line reaches the second passing point, for all of the passing points to be passed through; and
an evaluation step for evaluating a cognitive function of the subject based on the first inspection value data and the second inspection value data which have been extracted in the data extraction step.

2. The cognitive function evaluation method according to claim 1, wherein
in the data extraction step, position data related to positions of the first passing point and the second passing point as well as movement direction data related to a movement direction directed from the first passing point to the second passing point are extracted, from the inspection data obtained through the TMT execution step, for all the passing points to be passed through, and
in the evaluation step, the cognitive function of the subject is evaluated based on the first inspection value data, the second inspection value data, the position data, and the movement direction data which have been extracted through the data extraction step.

3. The cognitive function evaluation method according to claim 2, further comprising
a characteristic diagram generation step for generating a characteristic diagram in which the first inspection value data or the second inspection value data, and the position data or the movement direction data are displayed in association with each other, wherein
in the evaluation step, the cognitive function is evaluated based on the characteristic diagram generated in the characteristic diagram generation step.

4. The cognitive function evaluation method according to claim 1, wherein in the evaluation step, an MMSE score is estimated as a cognitive function evaluation value for the subject.

5. A cognitive function evaluation system that enables a TMT inspection on a display, evaluates a result of the inspection, and allows the result to be displayed on the display, comprising:
an inspection image generation circuit that electronically generates a TMT inspection image displayed on the display and obtained by setting passing points at a plurality of positions on a coordinate plane;
an inspection data acquisition circuit that acquires chronological data of a drawing trajectory drawn by a subject moving a contactor in a state of being in contact with a display surface of the TMT inspection image on the display so as to track the passing points in a predetermined order;
a data processing circuit that processes the data acquired by the inspection data acquisition circuit and makes a result of the processing displayable on the display as the result of the inspection; and
a control circuit that controls an operation of each of the circuits, wherein
the inspection data acquisition circuit includes a coordinate data acquisition circuit that acquires coordinate data corresponding to a position of the contactor on the coordinate plane based on a detection signal from a sensor that detects the contact of the contactor with the display surface of the TMT inspection image, and a time data acquisition circuit that acquires time data associated with an acquisition time of each piece of the coordinate data by a timer, and
the data processing circuit includes a computation circuit that computes, based on the coordinate data and the time data in the contactor, a first inspection value related to a residence time during which the contactor stagnates at a first passing point that is any one of the plurality of passing points from when the contactor reaches the first passing point until when the contactor starts moving to a second passing point to be passed through next, and a second inspection value related to a movement time required from when the contactor starts moving from the first passing point until when the contactor reaches the second passing point, for all of the passing points to be passed through, an evaluation circuit that evaluates a cognitive function of the subject based on the first inspection value and the second inspection value, and an inspection result outputting circuit that outputs the result of the inspection including a result of the evaluation executed by the evaluation circuit.

6. The cognitive function evaluation system according to claim 5, wherein
the inspection data acquisition circuit acquires position data related to positions of the first passing point and the second passing point as well as movement direction data related to a movement direction directed from the first passing point to the second passing point for all the passing points to be passed through, and
the evaluation circuit evaluates the cognitive function of the subject based on the first inspection value, the second inspection value, the position data, and the movement direction data.

7. The cognitive function evaluation system according to claim 6, wherein
the data processing circuit further includes a characteristic image generation circuit that generates a characteristic image in which the first inspection value or the second inspection value, and the position data or the movement direction data are displayed in association with each other, and an image output circuit that outputs the characteristic image generated by the characteristic image generation circuit, and
the evaluation circuit evaluates the cognitive function based on the characteristic image generated by the characteristic image generation circuit.

8. The cognitive function evaluation system according to claim 7, wherein the characteristic image generation circuit includes an identification image generation circuit that classifies the position data or the movement direction data into a plurality of groups based on the position or the direction on the coordinate plane, and generates the characteristic image in such a display form that inspection values corresponding to the respective groups are visually identifiable from one another.

9. The cognitive function evaluation system according to claim 8, wherein the computation circuit computes sums of the inspection values for the respective groups, and the identification image generation circuit generates a characteristic image that displays the sums for the respective groups.

10. The cognitive function evaluation system according to claim 8, wherein the computation circuit computes sums of the inspection values for the respective groups and computes rates of the sums of the respective groups with respect to a sum of all groups, and the identification image generation circuit generates a characteristic image that displays the rates for the respective groups.

11. The cognitive function evaluation system according to claim 5, further comprising a passage detection circuit that detects passage of the contactor at the passing point based on a detection signal from the sensor, wherein the passage detection circuit sets, for each passing point, a first coordinate region for determining that the contactor has passed through the passing point and a second coordinate region for determining that the contactor stagnates at the passing point, detects entry into the first coordinate region of the contactor as the passage of the contactor at the passing point, detects movement of the contactor in the second coordinate region as the stagnation of the contactor at the passing point, and detects movement of the contactor from the inside of the second coordinate region to the outside of the second coordinate region as the movement of the contactor from the passing point, and the computation circuit computes the first inspection value and the second inspection value based on signals indicating the stagnation and the movement from the passage detection circuit.

12. The cognitive function evaluation system according to claim 11, wherein the passage detection circuit includes a setting circuit for variably setting ranges of the first coordinate region and the second coordinate region.

13. The cognitive function evaluation system according to claim 5, wherein the evaluation circuit estimates an MMSE score as a cognitive function evaluation value for the subject.

14. The cognitive function evaluation system according to claim 5, wherein the control circuit controls an operation of each circuit based on an input signal from a mode selection menu that is displayed on the display and enables selection of an inspection form of a TMT inspection and a display form of a result of the inspection.

15. A computer program that enables a TMT inspection on a display, evaluates a result of the inspection, and allows the result to be displayed on the display,
the computer program causing a computer to execute:
an inspection image generating and displaying step for electronically generating a TMT inspection image obtained by setting passing points at a plurality of positions on a coordinate plane and displaying the TMT inspection image on the display;
an inspection data acquisition step for acquiring chronological data of a drawing trajectory drawn by a subject moving a contactor in a state of being in contact with a display surface of the TMT inspection image on the display so as to track the passing points in a predetermined order; and
a data processing and displaying step for processing the data acquired through the inspection data acquisition step and displaying a result of the processing on the display as the result of the inspection, wherein
the inspection data acquisition step includes a coordinate data acquisition step for acquiring coordinate data corresponding to a position of the contactor on the coordinate plane based on a detection signal from a sensor that detects the contact of the contactor with the display surface of the TMT inspection image, and a time data acquisition step for acquiring time data associated with an acquisition time of each piece of the coordinate data by a timer, and
the data processing and displaying step includes a computation step for computing, based on the coordinate data and the time data in the contactor, a first inspection value related to a residence time during which the contactor stagnates at a first passing point that is any one of the plurality of passing points from when the contactor reaches the first passing point until when the contactor starts moving to a second passing point to be passed through next, and a second inspection value related to a movement time required from when the contactor starts moving from the first passing point until when the contactor reaches the second passing point, for all of the passing points to be passed through, an evaluation step for evaluating a cognitive function of the subject based on the first inspection value and the second inspection value, and an inspection result outputting and displaying step for outputting the result of the inspection including a result of the evaluation in the evaluation step and displaying the result on the display.

16. The computer program according to claim 15, wherein
in the inspection data acquisition step, position data related to positions of the first passing point and the second passing point as well as movement direction data related to a movement direction directed from the first passing point to the second passing point are acquired for all the passing points to be passed through, and
in the evaluation step, the cognitive function of the subject is evaluated based on the first inspection value, the second inspection value, the position data, and the movement direction data.

17. The computer program according to claim 16, wherein
the data processing and displaying step further includes a characteristic image generation step for generating a characteristic image in which the first inspection value or the second inspection value, and the position data or the movement direction data are displayed in association with each other; and an image outputting and displaying step for outputting the characteristic image generated through the characteristic image generation step and displaying the characteristic image on the display, and
in the evaluation step, the cognitive function is evaluated based on the characteristic image generated through the characteristic image generation step.

18. The computer program according to claim 17, wherein the characteristic image generation step includes an identification image generation step for classifying the position data or the movement direction data into a plurality of groups based on the position or the direction on the coordinate plane, and generating the characteristic image in such a display form that inspection values corresponding to the respective groups are visually identifiable from one another.

19. The computer program according to claim 18, wherein in the computation step, sums of the inspection values are computed for the respective groups, and in the identification image generation step, a characteristic image that displays the sums is generated for the respective groups.

20. The computer program according to claim 18, wherein in the computation step, sums of the inspection values are computed for the respective groups and rates of the sums of the respective groups with respect to a sum of all groups are computed, and in the identification image generation step, a characteristic image that displays the rates is generated for the respective groups.

21. The computer program according to claim 15, which causes the computer to further execute a passage detection step for detecting passage of the contactor at the passing point based on a detection signal from the sensor, wherein in the passage detection step, a first coordinate region for determining that the contactor has passed through the passing point and a second coordinate region for determining that the contactor stagnates at the passing point are set for each passing point, entry into the first coordinate region of the contactor is detected as the passage of the contactor at the passing point, movement of the contactor in the second coordinate region is detected as the stagnation of the contactor at the passing point, and movement of the contactor from the inside of the second coordinate region to the outside of the second coordinate region is detected as the movement of the contactor from the passing point, and in the computation step, the first inspection value and the second inspection value are computed based on signals indicating the stagnation and the movement from the passage detection step.

22. The computer program according to claim 21, wherein the passage detection step includes a setting step for variably setting ranges of the first coordinate region and the second coordinate region.

23. The computer program according to claim 15, wherein in the evaluation step, an MMSE score is estimated as a cognitive function evaluation value for the subject.

24. The computer program according to claim 15, which enables a TMT inspection on the display and allows a result of the inspection to be displayed on the display based on an input signal from a mode selection menu that is displayed on the display and enables selection of an inspection form of the TMT inspection and a display form of the result of the inspection.

25. A cognitive function evaluation method that enables a TMT inspection on a display, evaluates a result of the inspection, and allows the result to be displayed on the display, the cognitive function evaluation method comprising:
an inspection image generating and displaying step for electronically generating a TMT inspection image obtained by setting passing points at a plurality of positions on a coordinate plane and displaying the TMT inspection image on the display;
an inspection data acquisition step for acquiring chronological data of a drawing trajectory drawn by a subject moving a contactor in a state of being in contact with a display surface of the TMT inspection image on the display so as to track the passing points in a predetermined order; and
a data processing and displaying step for processing the data acquired through the inspection data acquisition step and displaying a result of the processing on the display as the result of the inspection, wherein
the inspection data acquisition step includes a coordinate data acquisition step for acquiring coordinate data corresponding to a position of the contactor on the coordinate plane based on a detection signal from a sensor that detects the contact of the contactor with the display surface of the TMT inspection image, and a time data acquisition step for acquiring time data associated with an acquisition time of each piece of the coordinate data by a timer, and
the data processing and displaying step includes a computation step for computing, based on the coordinate data and the time data in the contactor, a first inspection value related to a residence time during which the contactor stagnates at a first passing point that is any one of the plurality of passing points from when the contactor reaches the first passing point until when the contactor starts moving to a second passing point to be passed through next, and a second inspection value related to a movement time required from when the contactor starts moving from the first passing point until when the contactor reaches the second passing point, for all of the passing points to be passed through, an evaluation step for evaluating a cognitive function of the subject based on the first inspection value and the second inspection value, and an inspection result outputting and displaying step for outputting the result of the inspection including a result of the evaluation in the evaluation step and displaying the result on the display.

26. The cognitive function evaluation method according to claim 25, wherein
in the inspection data acquisition step, position data related to positions of the first passing point and the second passing point as well as movement direction data related to a movement direction directed from the first passing point to the second passing point are acquired for all the passing points to be passed through, and
in the evaluation step, the cognitive function of the subject is evaluated based on the first inspection value, the second inspection value, the position data, and the movement direction data.

27. The cognitive function evaluation method according to claim 26, wherein
the data processing and displaying step further includes a characteristic image generation step for generating a characteristic image in which the first inspection value or the second inspection value, and the position data or the movement direction data are displayed in association with each other, and an image outputting and displaying step for outputting the characteristic image generated through the characteristic image generation step and displaying the characteristic image on the display, and
in the evaluation step, the cognitive function is evaluated based on the characteristic image generated through the characteristic image generation step.

28. The cognitive function evaluation method according to claim 27, wherein the characteristic image generation step includes an identification image generation step for classifying the position data or the movement direction data into a plurality of groups based on the position or the direction on the coordinate plane, and generating the characteristic image in such a display form that inspection values corresponding to the respective groups are visually identifiable from one another.

29. The cognitive function evaluation method according to claim 28, wherein in the computation step, sums of the inspection values are computed for the respective groups, and in the identification image generation step, a characteristic image that displays the sums is generated for the respective groups.

30. The cognitive function evaluation method according to claim 28, wherein in the computation step, sums of the inspection values are computed for the respective groups and rates of the sums of the respective groups with respect to a sum of all groups are computed, and in the identification image generation step, a characteristic image that displays the rates is generated for the respective groups.

31. The cognitive function evaluation method according to claim 25, causing a computer to further execute a passage detection step for detecting passage of the contactor at the passing point based on a detection signal from the sensor, wherein in the passage detection step, a first coordinate region for determining that the contactor has passed through the passing point and a second coordinate region for determining that the contactor stagnates at the passing point are set for each passing point, entry into the first coordinate region of the contactor is detected as the passage of the contactor at the passing point, movement of the contactor in the second coordinate region is detected as the stagnation of the contactor at the passing point, and movement of the contactor from the inside of the second coordinate region to the outside of the second coordinate region is detected as the movement of the contactor from the passing point, and in the computation step, the first inspection value and the second inspection value are computed based on signals indicating the stagnation and the movement from the passage detection step.

32. The cognitive function evaluation method according to claim 31, wherein the passage detection step includes a setting step for variably setting ranges of the first coordinate region and the second coordinate region.

33. The cognitive function evaluation method according to claim 25, wherein in the evaluation step, an MMSE score is estimated as a cognitive function evaluation value for the subject.

34. The cognitive function evaluation method according to claim 25, which enables a TMT inspection on the display and allows a result of the inspection to be displayed on the display based on an input signal from a mode selection menu that is displayed on the display and enables selection of an inspection form of the TMT inspection and a display form of the result of the inspection.

35. A cognitive function inspection method comprising:
a TMT execution step for executing an inspection based on a TMT for urging a subject to connect a plurality of passing points by a line such that the line passes through the passing points sequentially based on a predetermined rule;
a data extraction step for extracting, from inspection data obtained through the TMT execution step, first inspection value data related to a residence time during which a drawn line drawn by the subject sequentially tracking the passing points stagnates at a first passing point that is any one of the plurality of passing points from when the drawn line reaches the first passing point until when the drawn line starts moving to a second passing point to be passed through next, and second inspection value data related to a movement time required from when the drawn line starts moving from the first passing point until when the drawn line reaches the second passing point, for all of the passing points to be passed through; and
a data processing and displaying step for processing the first inspection value data and the second inspection value data which have been extracted in the data extraction step and displaying a result of the processing as an inspection result.

36. The cognitive function inspection method according to claim 35, wherein
in the data extraction step, position data related to positions of the first passing point and the second passing point as well as movement direction data related to a movement direction directed from the first passing point to the second passing point are extracted, from the inspection data obtained through the TMT execution step, for all the passing points to be passed through, and
in the data processing and displaying step, the first inspection value data, the second inspection value data, the position data, and the movement direction data which have been extracted through the data extraction step are processed and a result of the processing is displayed as an inspection result.

37. The cognitive function inspection method according to claim 36, further comprising
a characteristic diagram generation step for generating a characteristic diagram in which the first inspection value data or the second inspection value data, and the position data or the movement direction data are displayed in association with each other, wherein
in the data processing and displaying step, the characteristic diagram generated in the characteristic diagram generation step is displayed.
